(19) United States
(12) Patent Application Publication
(10) Pub. No.: US 2026/0022367 A1
(43) Pub. Date: Jan. 22, 2026

(54) BROAD SPECTRUM NANOZYMES

(71) Applicant: University of Florida Research Foundation, Inc., Gainesville, FL (US)

(72) Inventors: Yunwei Charles CAO, Gainesville, FL (US); Tian Jiang, Gainesville, FL (US)

(21) Appl. No.: 18/992,488

(22) PCT Filed: Jul. 18, 2023
(86) PCT No.: PCT/US2023/070377

§ 371 Date: Jan. 8, 2025

Related U.S. Application Data
(60) Provisional application No. 63/368,677, filed on Jul. 18, 2022.

(51) Int. Cl. C12N 11/14; A61K 9/51; C12N 9/22; C12N 15/113

(52) U.S. Cl. CPC C12N 11/14; A61K 9/5169; C12N 9/22; C12N 15/113

(57) ABSTRACT

Disclosed herein are improved broad-spectrum nanozymes for targeting RNA. The disclosed nanozymes are synthesized using recombinant ribonuclease with site-specific cysteine-substituted mutations that can be covalently functionalized with a length-tunable multithiol tether and then loaded onto gold particles through multiple gold-sulfur bonds, or inorganic particles with specific multiple ligand-to-particle-surface bonds. The disclosed nanozymes are also densely loaded with protective DNA oligonucleotides. In some embodiments, the disclosed nanozyme are core-free hollow forms. The removal of the inorganic nanoparticle cores from nanozymes can effectively eliminate the potential long-term toxicity induced by the core, and also creates a cavity for loading and delivery of small molecule drugs.

### CROSS-REFERENCE TO RELATED APPLICATIONS

This application claims benefit of U.S. Provisional Application No. 63/368,677, filed Jul. 18, 2022, which is hereby incorporated herein by reference in its entirety.

### STATEMENT REGARDING FEDERALLY SPONSORED RESEARCH OR DEVELOPMENT

This invention was made with Government Support under Grant No. 1710509 awarded by the National Science Foundation. The Government has certain rights in the invention.

### SEQUENCE LISTING

This application contains a sequence listing filed in ST.26 format entitled “222111-2930 Sequence Listing” created on May 19, 2023, having 8,473 bytes. The content of the sequence listing is incorporated herein in its entirety.

### BACKGROUND

RNA plays essential roles in living organisms. It translates genetic information into proteins forming the molecular machines and structures of cells, and RNA also regulates the activity of genes during development, cellular differentiation, and changing environments (Mello, C C, et al. Nature 2004 431 (7006): 338-42). Intervention of RNA metabolism can regulate the functions of genes and cells (Damase, T. R, et al. Front Bioeng Biotechnol 2021 9:628137). This provides an opportunity of using RNA as a target to develop therapeutic agents for treating human diseases (Feng, R, et al. Front Mol Biosci 2021 8:710738).

In the past forty years, two major therapeutical approaches have been developed to target RNA molecules in living cells: one is based on nucleic acids, and the other approach is based on ribonucleases. Nucleic-acid based approaches utilize antisense oligonucleotides, ribozymes, small-interfering RNAs or microRNAs to downregulate specific cellular mRNA through Watson-Crick base pairing (Hannon, G. J, et al. Nature 2004, 431 (7006): 371-8; Rakoczy, P. E. Methods Mol Med 2001 47:89-104; Thompson, J. D, et al. Nat Med 1995 1(3): 277-8). Small-interfering (si) RNAs and microRNAs can degrade sequence-specific mRNAs via formation of RNA-induced silencing complex (RISC) through the RNA inference (RNAi) pathway (Moazed, D. Nature 2009 457 (7228): 413-20). Due to its high efficacy, RNAi technology has been used as the primary method for controlling mRNA levels in routine biomedical research (Kim, D. H, et al. Nat Rev Genet 2007 8(3): 173-84). Nucleic-acid based approaches also include the methods of direct delivery of mRNAs into cells to synthesize specific proteins in vivo, which can revolutionize vaccination, protein replacement therapies, and the treatment of genetic diseases (Copur, M., Oncology 2021 35 (4): 190-198; Van Hoecke, L, et al. Mol Cancer 2021 20 (1): 48). To date, a number of antisense RNA drugs and siRNA drugs have been proved for clinical use by FDA (Van Hoecke, L, et al. Mol Cancer 2021 20 (1): 48; Banerji, A, et al. J Allergy Clin Immunol Pract 2021 9 (4): 1423-1437). Very recently, mRNA COVID-19 vaccines have been applied worldwide and proven to be crucial in stopping the pandemic (Pawlowski, C, et al. Med (N Y) 2021 2 (8): 979-992. e8).

Besides nucleic-acid based approaches, approaches of using toxic ribonucleases (RNases) have also been extensively investigated for therapeutic applications (Arnold, U, et al. Biotechnol Lett 2006 28 (20): 1615-22; Gundampati, R. K, et al. J Mol Model 2012 18 (2): 653-62). RNase-based approaches do not rely on Watson-Crick base pairing. Ribonucleases can degrade a broad spectrum of RNA molecules in a non-sequence specific manner. Ribonucleases from RNase A superfamily have long been recognized as a crucial part of host defense system against bacterial and viral pathogens (Ilinskaya, O. N, et al. Mol Biol 2014 48 (5): 615-623; Li, J, et al. Virulence 2021 12 (1): 444-469; Kosgey, J. C, et al. Int J Biol Macromol 2020 160:1042-1049). RNase 1 extracts from human urine as well as recombinant RNase 1 showed antiviral activity against human immunodeficiency virus (HIV)-1 (Bedoya, V. I, et al. AIDS Res Hum Retroviruses 2006 22 (9): 897-907; Koczera, P, et al. Int J Mol Sci 2016, 17 (8)). It has been found that RNase 1 plays an important role in normalization of serum viscosity and clearance of perivascular pathogenic polynucleotides (Landré, J. B, et al. J Cell Biochem 2002 86 (3): 540-52).

With its capability of RNA degradation, RNases are important alternatives to the conventional DNA damaging chemotherapeutics and broad-spectrum intracellular exogenous RNA degradation reagents in fighting cancers, as well as viral or other pathogen infections (Arnold, U, et al. Biotechnol Lett 2006 28 (20): 1615-22; Makarov, A. A, et al. FEBS Lett 2003 540 (1-3): 15-20). Early in 1950s, RNase A has been demonstrated to be effective in killing tumor cells, both in vitro and in vivo, under extremely high dosages (injection of milligrams of RNase A into solid tumors) (Ledoux, L., Biochimica Et Biophysica Acta 1956 20 (2): 369-377; Easty, D, et al. Biochimica Et Biophysica Acta 1956 20 (3): 528-537; Ledoux, L., Nature 1955, 175 (4449): 258-259; Telford, I. R, et al. Proc Soc Exp Biol Med 1959 100 (4): 829-31). In 1970s, RNase A was tested for its potential antitumor effect clinically. Although no therapeutic efficiency was detected for most of the patients involved in the study, in a few cases, significant tumor regression or even disappearance was observed, again with large amount of RNase A being used directly to tumors (milligrams of RNase A per kg body weight per day) (Ardelt, W, et al. European Journal of Pharmacology 2009 625 (1-3): 181-189). One of the most important reasons for the requirement of such high dosage was the inactivation of RNase catalytic activity by RNase inhibitors (RI) in cytosol (Dickson, K, et al. Prog Nucleic Acid Res Mol Biol. 2005 80:349-74). RI, which can be found in all mammalian cells, controls the activity of all RNases in different ways (Shapiro, R. Ribonucleases, Pt a 2001 341:611-628). The RI binds to ribonucleases with femtomolar affinity and inhibits the biological effects of the RNases by generating an RNase: RI complex (Dickson, K, et al. Prog Nucleic Acid Res Mol Biol. 2005 80:349-74). The presence of cytosolic RI protects the host cells from the cytotoxic activity of RNases.

The ability of evading cytosolic RI is a prerequisite for RNase drugs to exhibit therapeutic effects against cancer cells or pathogenic infections (Leland, P. Chemistry & Biology 2001 8 (5): 405-413). Onconase (a RNase A variant isolated from Northern Leopard Frog) exhibits ability to evade the cytosolic RI and degrades cellular RNA (Ardelt, W, et al. Current Pharmaceutical Biotechnology 2008 9 (3): 215-225). Onconase has been evaluated clinically for its antitumor activity but did not meet statistical significance for the primary endpoint in phase III clinical trial (Ardelt, W, et al. J Biol Chem 1991 266 (1): 245-51; Lee, J, et al. Biodrugs 2008 22 (1): 53-58). Currently, Onconase is in clinical trial for treating human papilloma virus (Vert, A, et al. Oncotarget 2017 8 (7): 11692-11707). However, with its nonmammalian origin, the potential immunogenicity is always a concern. Through site directed mutagenesis and proper chemical modification, Raines et al have developed a number of mammalian RNase A mutants capable of evading RI (Leland, P, et al. Proc Natl Acad Sci USA. 1998 95 (18): 10407-12). By replacing Gly88 in RNase A with Arg or Asp, the mutant RNases were found to be about 1000-fold less affinitive to RI and exhibited cytotoxicity of sub 10 μM IC₅₀ (Rutkoski, T. J, et al. Bioconjug Chem 2010 21 (9): 1691-702; Lomax, J. E, et al. Methods Enzymol 2012 502:273-90). In addition, QBI-139, an RI-evading RNase 1 mutant (human homolog of RNase A), is under phase I clinical trial for treating solid tumors (Strong, L, et al. Journal of Clinical Oncology 2011 29 (15)).

### SUMMARY

This disclosure describes the design, synthesis, and applications of a new class of nanozymes, referred to herein as broad-spectrum nanozymes, which can actively degrade intracellular RNAs in the presence of RNase inhibitors in a non-sequence specific manner. These broad-spectrum nanozymes exhibit high enzymatic activities in RNA degradation and high cellular-uptake rates. These nanozymes can effectively manipulate the functions of living cells by catalyzing the cleavage of a broad spectrum of cellular RNAs, such as exogenous RNAs, tRNA, rRNA, mRNA as well as the non-coding RNAs. These broad-spectrum nanozymes display excellent antiviral efficacy against hepatitis C virus in cultured cells, and strong anticancer effects on a verity of human tumor cell lines (such as A549, Hela, K562, Ramos, CCRF-Cem cells). In addition, by incorporating traffic-guiding moieties, the broad spectrum nanozymes can be endowed with selective cellular entry properties, thus elevating their target cell specificity. Also disclosed herein is the design, synthesis, and characterization of broad spectrum nanozymes in a core-free hollow form.

Broad-spectrum nanozymes can be synthesized in two steps: firstly, multi-thiol functionalized RNases can be loaded onto Au nanoparticles with various loading densities to optimize the activity. Secondly, single-sequenced capturer DNA without binding specificity to any RNA under physiological conditions can be densely loaded onto the surface of Au nanoparticles. Moreover, both spacers of RNase and capture DNA can be derivatized with polymerizable moieties to enable the synthesis of hollow broad-spectrum nanozymes. Additional guiding moieties can also be attached onto the capturer DNA for the preparation of broad-spectrum nanozymes possessing specific cellular entry.

Importantly, these broad-spectrum nanozymes exhibit cellular-uptake rates of more than one order of magnitude higher than those sequence-selective nanozymes, and they display very high RNase catalytic activity in RNA cleavage. As compared with Onconase and QBI-139, which are being evaluated in clinical anticancer trials, broad-spectrum nanozymes exhibit much more potent toxicity toward various cancer cell lines, such as, A549 cells, K-562 cells, CCRF-CEM cells, Ramos cells, and Hela cells. For example, experimental results have shown that broad-spectrum nanozymes can display a IC₅₀ values of 10.7 nM against A549 cells. In the similar conditions, onconase and QBI-139 showed IC₅₀ values of 106 μM and 62 μM (Hoang, T, et al. Molecular Cancer Therapeutics 2018 17 (12): 2622-2632; Lee, I, et al. Anticancer Research 2007 27 (1A): 299-307), respectively.

The disclosed results suggest that nanozyme's potent cytotoxicity is due in a large part to their ability to evade cytosolic RI, their high cellular uptake rates and high RNase catalytic activity. These nanozyme properties are tailorable through varying the length and their loading density of single-stranded DNA oligonucleotides on nanoscopic surface, and the loading density of RNase. The presence of DNA oligonucleotides at close approximate to RNase in nanoscopic surfaces are essential for nanozyme's ability to evade RI inhibition. When DNA oligonucleotides are replaced with polyethylene glycol in nanozymes, RNase catalytic activity in RNA cleavage can be totally inhibited by RI. Nanozyme's RI-evasion ability is not strongly dependent on the length of DNA oligonucleotides and the loading density of RNase, but their cellular uptake rates and RNase catalytic activity are sensitively dependent on these parameters. The shorter the DNA oligonucleotide is, the higher the cellular uptake rates and RNase catalytic activity, and vice versa.

The disclosed results have further shown that broad-spectrum nanozymes can effectively degrade ribosomal RNAs inside cells, and higher cellular uptake rates and RNase catalytic activity are associated with higher nanozyme's cytotoxicity and lower IC₅₀ values. Broad-spectrum nanozymes exhibit higher toxicity toward cancer cells than noncancer cells. Detailed mechanisms for this favorable therapeutic index have yet been fully understood clear. This preferential toxicity would be related to some unique biological processes of cancer cells. The rapid proliferation of cancer cells and/or some cancer survival and growth pathways make them more reliant on the integrity of their RNA (Dong, C, et al. Biochem Biophys Res Commun 2016 476 (4): 340-345; Drygin, D, et al. Cancer Res 2009 69 (19): 7653-61). These factors should be dependent on cancer cell lines. Indeed, under similar conditions, broad-spectrum nanozymes showed higher toxicity toward Hela cells than A549 cells, displaying IC₅₀ values of 6.06 nM and 10.7 nM, respectively.

When their dose (or concentration) is lower IC₅₀ values, broad-spectrum nanozymes still have interference effects on biological pathways inside cells. They displayed potent effects onto the pathways associated with exogenous RNAs than those with endogenous RNAs. For examples, broad-spectrum nanozymes displayed potent antiviral efficacy against hepatitis C virus in cultured cells. This antiviral effect is likely achieved via a competitive mechanism, where endogenous RNAs can be regenerated from cellular genomic transcriptions, but exogenous RNAs cannot. This opens an opportunity of using broad-spectrum nanozymes as effective therapeutic agents to treat infections of RNA virus, such as HCV and COVID-19. In addition, additional guiding moieties can be introduced onto nanozymes to make their function specific to chosen cell types or cells bearing specific biomarkers. Broad-spectrum nanozymes functionalized with specific endocytosis guiding moieties can target cells selectively.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1. Schematic representation of the designing of multi-thiol functionalized RNase A and RNase 1 (a). All enzyme mutants are linked to a multi-DTPA (dithiol phosphoramidite) or lipoamido (dithiol lipoic acid moiety) terminated PEG spacer via click chemistry. Specific structures are depicted. Amino acid sequences of RNase A (SEQ ID NO:1), A19C (SEQ ID NO:2), G88C (SEQ ID NO:3), RNase 1 (SEQ ID NO:4), P19C (SEQ ID NO:5), and G89C (SEQ ID NO:6) (Leu, Y. J, et al. J Biol Chem 2003 278 (9): 7300-9) (b). Mutated amino acids are marked by arrows.

FIG. 2. Schematic representation describing the synthesis procedures of broad-spectrum nanozyme (a), and sequence-selective nanozyme (b) is used as a control in this study.

FIG. 3. Schematic representation describing the synthesis procedures of broad-spectrum nanozyme with guiding moieties.

FIG. 4. Schematic representation describing the synthesis procedures of hollow broad-spectrum nanozyme (a), sequence-selective nanozyme (b) which is used as a control in this study, and crosslinking mechanism of propargyl groups on the surface of Au nanoparticles (c) (Zhang, K, et al. J Am Chem Soc 2010 132 (43): 15151-3).

FIG. 5. Schematic representation describing the synthesis procedures of porous hollow broad-spectrum nanozyme (a), drug molecules-encapsulated hollow broad-spectrum nanozyme (b).

FIG. 6. A19C/G88C mutant RNase A, as well as P19C/G89C mutant RNase 1 expression and purification procedures.

FIG. 7. Reaction of thiol capping with 5,5′-dithiobis (2-nitrobenzoic acid).

FIG. 8. Typical fast protein liquid chromatography chromatogram of A19C (a), G88C (b) RNase A.

FIG. 9. Tagging of A19C/G88C RNase A, or P19C/G89C RNase 1 with lipoic acid moiety via copper free click chemistry.

FIG. 10. Example MALDI-TOF spectrum of A19C (a) and LA-A19C (b) via copper free click chemistry. The theoretical m/z of A19C and LA-A19C were 13803 and 14753, and the observed m/z were 13747 and 14690.

FIG. 11. Tagging of Cysteine-containing mutant RNase with lipoic acid moiety via copper assisted click chemistry.

FIG. 12. Example MALDI-TOF spectrum of A19C (a) and LA-A19C (b) via copper assisted click chemistry. No m/z change were observed after reaction.

FIG. 13. Hydrolyzing of cytidine 2′,3′-cyclic monophosphate by RNase A/RNase 1.

FIG. 14. Reaction of primary amine with fluorogenic 3-(4-carboxybenzoyl) quinoline-2-carboxaldehyde.

FIG. 15. Ribonuclease activity tests for assessing the RI-resistance of (A) broad-spectrum nanozyme with protecting DNA composed of an A₆ strand with a PEG-spacer and various RNase loading density, (B) Au-RNase-PEG NPs of identical RNase loading.

FIG. 16. (a) Cell viability of A549 cells treated with nanozymes of varied LA-A19C and DNA loading density for 48 h. (b) Cell viability of A549 cells treated with 20 nM of Au-LA-A19C-PEG500 NPs (same IA-A19C loading density as nanozyme6), Au-DNA NPs, Au-PEG NPs, or 5 μM free RNase A for 48 h.

FIG. 17. Cellular uptake of nanozymes and Au-LA-A19C-PEG500 NPs of same LA-A19C loading density, and Au-poly A₆ NPs without LA-A19C. A549 cells were treated for 1 nM NPs for 48 h before analysis. Cellular uptake of Au-LA-A19C-PEG500 NP1 and 2 were not measured since serious aggregation was noticed in cell culture medium.

FIG. 18. Evaluation of RNase activity and RI-resistance for nanozymes prepared from oligonucleotides of different length.

FIG. 19. Cell viability of A549 cells treated with nanozymes of poly A₆, DNA1 (18 bases) and DNA2 (39 bases) for 48 h.

FIG. 20. Cellular uptake of nanozymes with oligonucleotides of varied length (poly A₆, anti-HCV DNA1 and DNA2 of 18 and 39 bases, respectively) and the corresponding Au-oligonucleotides NPs without LA-A19C, expressed as average number of Au NPs within one cell. A549 cells were treated for 1 nM NPs for 48 h before analysis.

FIG. 21. Evaluation of (a) time-dependent (1 nM nanozyme) and (b) concentration-dependent (12 h incubation) endocytosis of nanozyme (53 LA-A19C and 54 poly A₆ molecules per Au NP, A549 cells were treated for 1 nM nanozyme for analysis).

FIG. 22. Evaluation of (a) time-dependent (53 LA-A19C and 54 poly A₆ per Au NP, A549 cells were treated for 20 nM nanozyme for analysis) and (b) concentration-dependent (53 LA-A19C and 54 poly A₆ per Au NP, A549 cells were treated for 48 h for analysis) cytotoxicity of nanozyme. The data was fitted into with four parameter logistic model (Yu, L, et al. Molecules 2019 24 (21)) using Origin software (fitting line).

FIG. 23. Agarose gel electrophoresis of A549 cells intracellular RNA treated with varied concentrations of nanozyme for different time.

FIG. 24. Electrophoresis (TBE-urea gel) of A549 cells intracellular RNA treated with varied concentrations of nanozyme for different time.

FIG. 25. Evaluation of concentration-dependent cytotoxicity of nanozyme (53 LA-A19C and 54 poly A₆ per Au NP, cells were treated for 48 h for analysis) on (a) HeLa, (b) K562, (c) CCRF-CEM and (d) Ramos cells. The data was fitted into with four parameter logistic model (Yu, L, et al. Molecules 2019 24 (21)) using Origin software (fitting line) for determination of IC₅₀.

FIG. 26. Agarose gel electrophoresis of intracellular RNA of A549, HeLa, K562, CCRF-CEM and Ramos cells treated with nanozyme at IC₅₀.

FIG. 27. Electrophoresis (TBE-urea gel) of intracellular RNA of A549, HeLa, K562, CCRF-CEM and Ramos cells treated with nanozyme at IC₅₀.

FIG. 28. Transmission electron microscope image of hollow broad-spectrum nanozymes with uranium acetate negative staining.

FIG. 29. Ribonuclease activity tests for assessing the RNase activity and RI-resistance of hollow broad-spectrum nanozymes.

FIG. 30. qRT-PCR analyses of HCV RNA expression in the Huh 7.5 cells harboring JFH1 HCV RNA replicon treated with control NPs and nanozymes at varying doses for 48 h. The relative HCV RNA level was 32% and 16% for cell treated with 1 and 5 nM sequence-selective nanozymes; 34% and 17% for cells treated with 1 and 5 nM hollow sequence-selective nanozymes; 8.1% and 1.1% for cells treated with 1 and 5 nM broad-spectrum nanozymes; 9.0% and 2.3% for cells treated with 1 and 5 nM hollow broad-spectrum nanozymes; Student's t test: ns for non-significance: P>0.14, * for P<0.01, and ** for P<0.001.

FIG. 31. Cell viability of Huh7. 5 cells harboring JFH1 HCV replicons treated with control NPs and nanozymes for 48 h.

FIG. 32. Cellular uptake of broad-spectrum nanozymes (a) and sequence-selective nanozymes (b) into Huh7. 5 cells harboring JFH1 HCV replicons, expressed as average number of NZs within one cell. Cells were treated for 1 nM NPs for 48 h before analysis.

FIG. 33. Agarose gel electrophoresis of intracellular RNA of Huh7. 5 cells harboring JFH1 HCV replicons. Cells were treated with broad-spectrum nanozymes (a) or sequence-selective nanozymes (b) for 48 h and analyzed, or first treated with nanozyme for 48 h and then recovered in fresh medium without nanozyme for 48 h and analyzed. Since the amount of HCV viral RNA was far less than that of ribosomal RNA, band of HCV viral RNA was not observable on agarose gel.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e. g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

### Nanozymes

Embodiments of the present disclosure provides for nanozymes, methods of making nanozymes, methods of using nanozymes, and the like. In an embodiment, the nanozyme can include a gold nanoparticle, an enzyme, and a recognition moiety. Each of the enzyme and the recognition moiety are attached (e. g., directly or indirectly via a linker (e. g., compound or protein) or the like) to the nanoparticle by gold-sulfur bonds.

### Kits

This disclosure encompasses kits, which include, but are not limited to, nanozymes, and directions (written instructions for their use). The components of the nanozyme can be tailored to the particular disease, condition, or even being studied and/or treated. The kit can further include appropriate buffers and reagents known in the art for administering various combinations of the components listed above to the host cell or host organism.

### Delivery Methodology

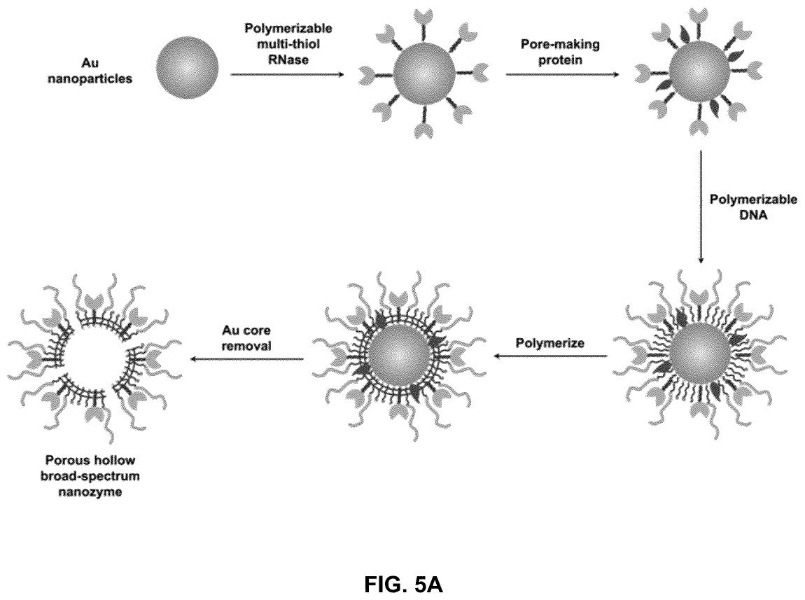

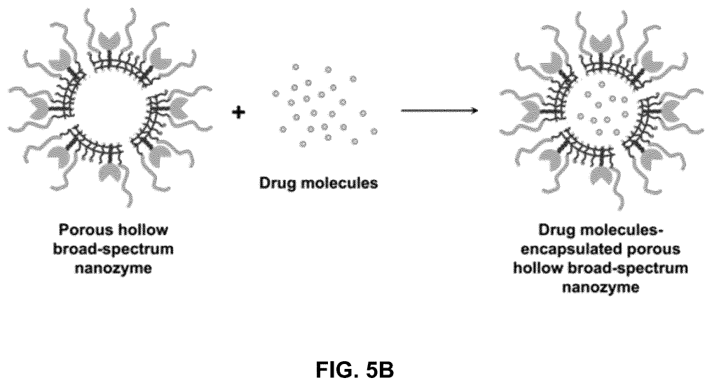

Broad-spectrum nanozyme could be delivered in various methods, including intravenous/locally injection, and pulmonary delivery. Nanoparticle-based nanomedicine has long been recognized as promising candidates of anti-cancer/viral therapeutics (Delcardayre, S, et al. Protein Engineering 1995 8 (3): 261-273; Kobe, B, et al. Journal of Molecular Biology 1996 264 (5): 1028-1043). Capable of both actively targeting specific cells and tissues through the guiding moieties on their surface (as discussed in C3) or passively via the enhanced permeation and retention (EPR) effect (Rosi, N, et al. Science 2006 312 (5776): 1027-1030; Shen, R, et al. Acta Biochim Biophys Sin (Shanghai) 2016 48 (10): 894-901), broad-spectrum nanozymes possess remarkably enhanced selectivity against cancer cells/viral infection with reduced toxicity to normal cells. Through intravenous injection, broad-spectrum nanozymes could be readily circulated to specific areas of body, inducing fast therapeutic responses (Chao, T. Y, et al. Biochemistry 2011 50 (39): 8374-82). Moreover, for certain types of diseases related with circulatory system, such as leukemia and viral infection of blood cells, direct intravenous injection renders broad-spectrum nanozyme therapeutically effective right upon administration, providing almost instantaneous response (Chao, T. Y, et al. Biochemistry 2011 50 (39): 8374-82; Haigis, M. C, et al. J Cell Sci 2003 116 (Pt 2): 313-24). Besides intravenous injection, localized injection of broad-spectrum nanozyme into disease area, such as tumors, offers another option of tumor size reduction and inhibition, together with tumor removal surgery, improving overall curing rate. Other than injection, pulmonary delivery of broad-spectrum nanozymes, such as oral inhalation, enables effective therapeutic administration against respiratory diseases, including COVID-19 and lung cancer. Compared with injection administration, pulmonary delivery offers advantages of avoiding first-pass hepatic metabolism and rapid absorption of nanozymes though large surface areas of vascularization. Moreover, as discussed in section C5, porous hollow broad-spectrum nanozymes are capable of encapsulating small molecule drugs (FIG. 5B), therefore further boosting their therapeutic efficiency.

A number of embodiments of the invention have been described. Nevertheless, it will be understood that various modifications may be made without departing from the spirit and scope of the invention. Accordingly, other embodiments are within the scope of the following claims.

### Examples

### Example 1: Synthesis of Broad-Spectrum Nanozymes

A nanozyme technology was previously developed that utilize both nucleic acids and ribonucleases (Wang, Z, et al. Proc Natl Acad Sci USA. 2012 109 (31): 12387-12392). A nanozyme is synthesized by co-assembly of single-stranded DNA oligonucleotides and ribonucleases onto a nanoscopic surface using gold nanoparticles as scaffolds. These gold nanoparticle scaffolds can be removed after synthesis to avoid potential toxicity during long-term treatments (Sharma, V. K, et al. Chem Soc Rev 2015 44 (23): 8410-23). In this nanozyme, single-strand DNA oligonucleotides with a designed sequence bind with target RNAs through Watson-Crick base pairing, and then direct the neighboring ribonucleases to enzymatically cleave captured RNA molecules. These nanozymes evade cytosolic RI and exhibit extraordinary functional stability in the presence of proteinases and DNases.

Unlike siRNA drugs which rely on special delivery vehicles for cellular entry, nanozymes exhibit efficient cellular uptake without any additional physical or chemical transfecting reagents (Whitehead, K. A, et al. Nat Rev Drug Discov 2009 8 (2): 129-38). Moreover, the functions of nanozymes are independent on any assistance of other intracellular enzymes or pathways, therefore avoiding the risk of interfering the natural intracellular gene regulation machineries (Castanotto, D, et al. Nature 2009 457 (7228): 426-433; Grimm, D, et al. Nature 2006 441 (7092): 537-541). With carefully designed capturer DNA and site-specific engineered RNase A, it was shown that nanozymes can display excellent efficacy in selective degradation of target mRNAs in cultured cells such as hepatitis C virus (HCV) RNA, Ebola RNA and GPC3 RNA. In effective dose (or concentration) range, these nanozymes exhibit nearly no cytotoxicity and do not trigger innate immune responses. There was a 99.7% decrease in HCV virus RNA levels in mice models treated with nanozymes.

The length of single stranded DNA oligonucleotides and their loading density on nanoscopic surface are important parameters in defining the functions and biochemical properties of these sequence-selective nanozymes: RNA target sequence specificity, RNase enzymatic activity, cytotoxicity, the ability to evade cytosolic RI, and the abilities to resist enzymatic digestions of proteinases and DNases. Here, it is shown that reducing the length and/or surface loading density of single-stranded DNA oligonucleotides can significantly decrease nanozyme's RNA sequence specificity. In some DNA-oligonucleotide loading configurations, nanozymes totally lose their RNA sequence specificity, but still retain their ability to evade cytosolic RI, and abilities to resist enzymatic digestions of proteinases and DNases. These sequence non-specific nanozymes are referred to herein as “broad-spectrum nanozymes”.

Preparation of Recombinant RNase with a Multi-Thiol Modified Tether

### Production of RNase Mutant.

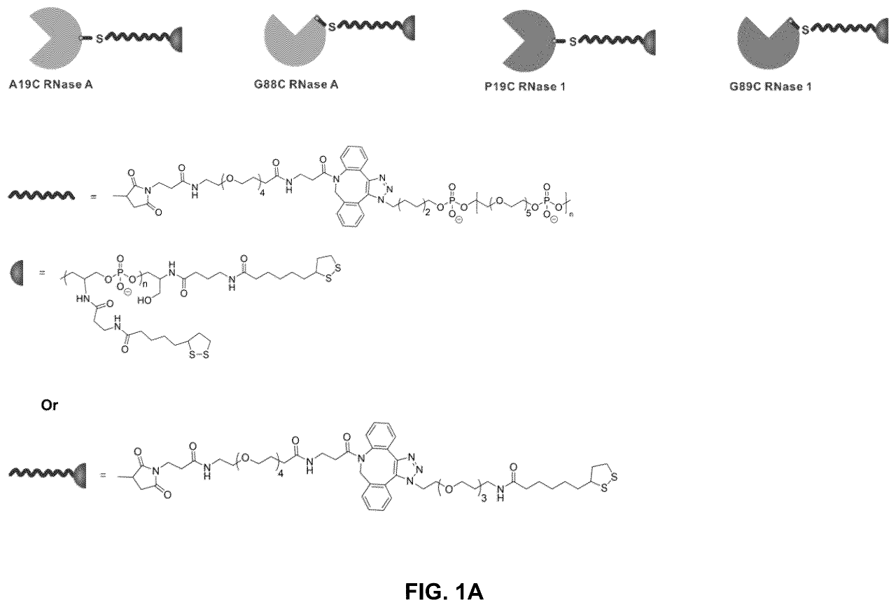

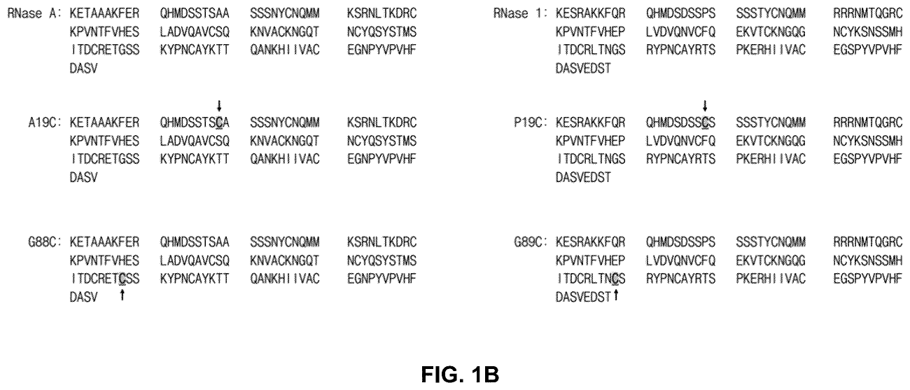

Suitable RNase mutants were prepared for functionalization. Because of the well-established production procedures and relatively high enzymatic activities as compared with the wildtype RNase, four mutants were chosen as model enzymes for nanozyme construction, A19C/G88C RNase A (Ala19 and Gly88 are mutated into cysteine), and P19C/G89C RNase 1 (Pro19 and Gly89 are mutated into cysteine) (Rutkoski, T, et al. Cancer Biology & Therapy 2011 12 (3): 208-214; Lomax, J. E, et al. Biochem J 2017 474 (13): 2219-2233; Rutkoski, T. J, et al. Transl Oncol 2013 6 (4): 392-7). Ala19 of RNase A and Pro19 of RNase 1 are on the back of the active site, while Gly88 of RNase A and Gly89 of RNase 1 are close to the active site (FIG. 1). The thiol groups on these RNase mutant molecules enable precise multi-thiol functionalization at these locations.

According to well-established procedures (Leland, P, et al. Proc Natl Acad Sci U SA. 1998 95 (18): 10407-12; Rutkoski, T, et al. Cancer Biology & Therapy 2011 12 (3): 208-214), these RNase mutants were expressed using pET22b(+)/pET27b(+) plasmid and Escherichia coli (E. coli) BL21 (DE3) cells. All the solutions involved for inclusion bodies dissolving and buffers for enzyme refolding were purged with N₂ to prevent the possible oxidation of free thiol groups. 5,5′-dithiobis(2-nitrobenzoic acid) was used for thiol protection and the protein was stored at −80° C. after further fast protein liquid chromatography (Leland, P, et al. Proc Natl Acad Sci USA. 1998 95 (18): 10407-12; D′Avino, C, et al. Protein Eng Des Sel 2014 27 (3): 83-8).

### Preparation of Multi-Thiol Functionalized RNase

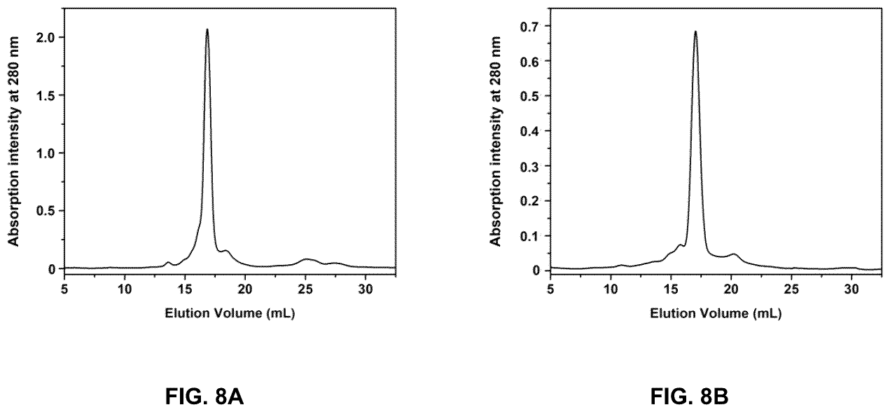

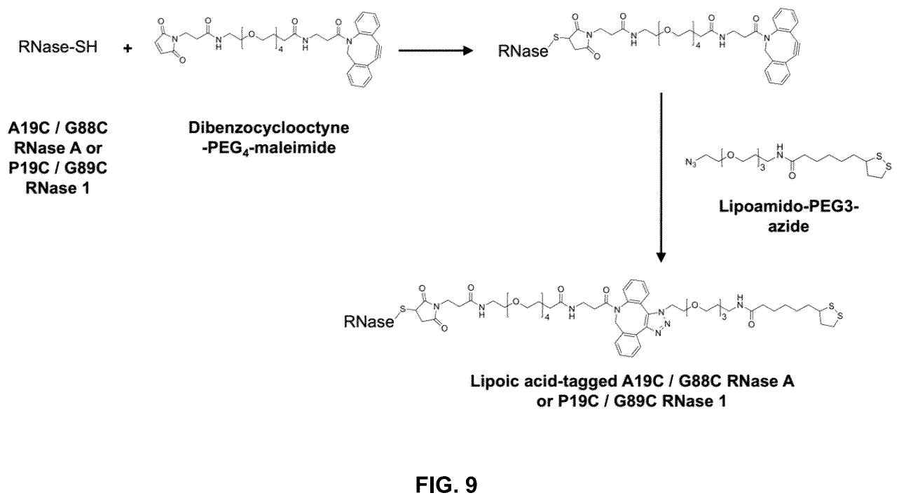

Multi-thiol functionalized RNase was constructed. As shown FIG. 9, multi-thiol functionalized RNase is prepared as follows. The enzyme mutants were firstly reacted with dibenzylcyclooctyne-PEG₄ (detailed structures shown in FIG. 9)-maleimide to introduce a dibenzylcyclooctyne group for cross-linking via click chemistry, then these dibenzylcyclooctyne-containing enzymes were reacted with lipoamido-PEG₃-azide (detailed structures shown in FIG. 8), resulting in lipoamido functionalized RNase. For introducing more than 2 thiols per one RNase, the PEG tether was synthesized using a solid-state oligonucleotide synthesizer applying 3′-dithiol serinol CPG, 5′-bromohexyl phosphoramiditem and spacer phosphoramidite 18, followed by 5′-bromide into an azide conversion using sodium azide. Then, the multi-thiol terminated PEG was linked to dibenzylcyclooctyne-functionalized RNase via click chemistry. All the reagents mentioned above are commercially available and reactions are robust and easy. The constructed RNase functionalized with multi-thiol moiety were purified by cation exchange chromatography and examined on their enzymatic activity applying a small RNA substrate cytidine 2′,3′-cyclic monophosphate, following established procedures (Crook, E, et al. Biochemical Journal 1960 74:234-238).

### Preparation and Characterization of Broad-Spectrum Nanozymes

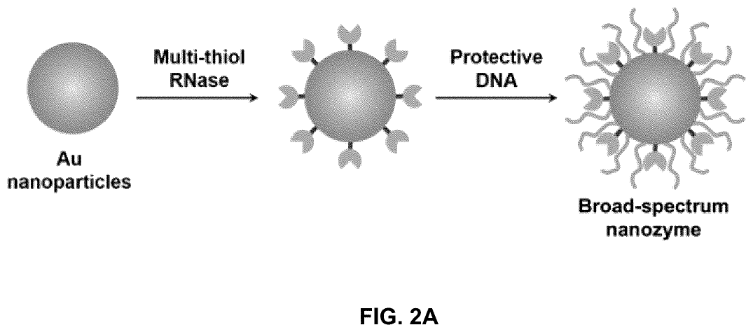

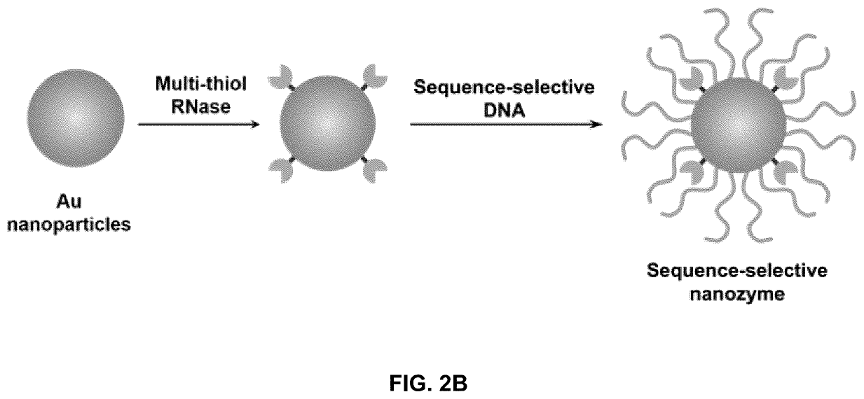

Broad-spectrum nanozyme was prepared and characterized. Generally, broad-spectrum nanozyme can be prepared via a two-step synthesis (FIG. 2A). In the first step, multi-thiol modified RNase was loaded onto Au nanoparticles. The loading conditions of multi-thiol functionalized RNase were optimized to ensure the formation of Au-RNase complex with specific Au-thiol binding and correct orientation of enzyme catalytic sites. Wild type RNase was used as model enzymes and various small molecule additives, such as Tween 20 and citrate, was used to suppress the nonspecific binding between RNase and Au nanoparticles. Secondly, protective DNA ligands were densely loaded onto the surface of Au nanoparticles.

During the first step of RNase loading, various enzyme/Au nanoparticle ratios were carried out and the corresponding enzymatic activity of Au-RNase complex was tested using agarose gel electrophoresis and compared with that of free RNase mutant, RNase mutant after multi-thiol functionalization, and wild type RNase. Due to the conformational constraint and lack of freedom, Au-RNase complex may exhibit significantly lowered enzymatic activity. Under such circumstances, the length of PEG tether between RNase and thiol anchor was increased to render the enzyme more conformationally free so as to improve its enzymatic activity. Considering the functionalization position of four mutants, A19C RNase A and P19C RNase 1 may show better activity with a relatively short PEG tether since its catalytic pocket will be exposed toward the outside (FIG. 1).

Capture DNA molecules in a high surface loading density are essential for nanozymes' RI resistance. Therefore, during the second step of broad-spectrum nanozyme preparation, the length and loading density of DNA strands on RNase-NP were carefully tuned, and their effects on nanozymes' enzymatic activity and RI-resistance were examined by agarose gel electrophoresis. To quantify the number of RNase and DNA strands on each nanozyme, as-prepared nanozymes were dissolved using a KCN solution (0.05 M) to remove the Au nanoparticle backbone, and RNase and DNA strands per Au nanoparticle were quantified using commercial fluorescence protein and DNA quantification kits.

### Preparation and Characterization of Broad-Spectrum Nanozymes Guiding Moieties

The ability to design and synthesis broad-spectrum nanozyme with guiding moieties was explored. Such design endows nanozymes with selective binding and entry to target cells, which are of great significance for further elevating nanozymes' therapeutic efficiency and reducing any possible side effects. The proposed traffic guiding moieties could be small molecule ligands or peptides for cell membrane receptors, or biomacromolecules such as aptamer or antibodies presenting specific binding to certain types of cells.

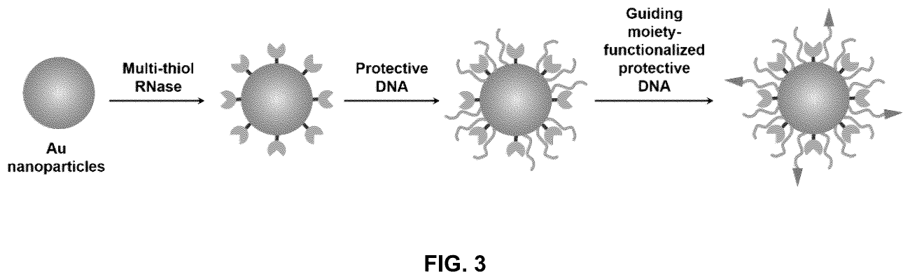

Generally, broad-spectrum nanozyme with guiding moieties were prepared in three steps (FIG. 3). First, multi-thiol modified RNase was loaded onto Au nanoparticles. Then, protective DNA was loaded in the second step. However, by controlling the Au/protective DNA ratio, the loading of protective DNA on Au nanoparticles is not as full as normal broad-spectrum nanozyme. In the third step, the resulting nanozymes were further functionalized with guiding moieties-functionalized protective DNA for maximizing the DNA coverage on Au nanoparticles and adding guiding moieties for selectively targeting a chosen cell type or cells on/in a chosen tissue.

### Preparation and Characterization of Hollow Broad-Spectrum Nanozymes

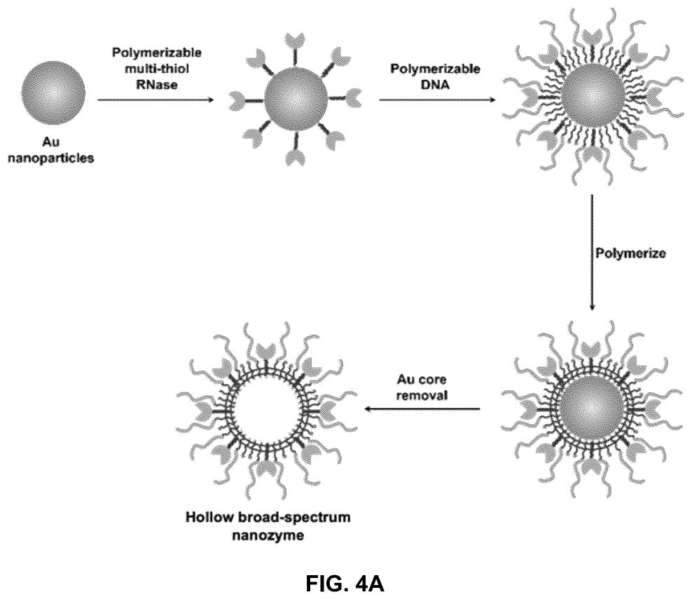

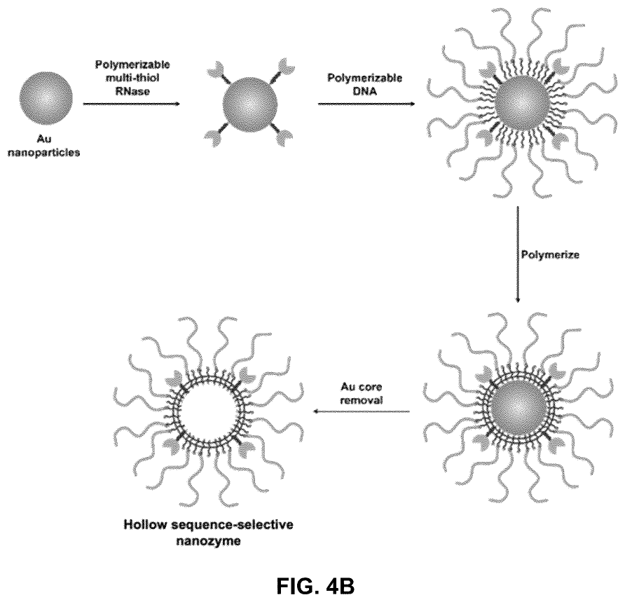

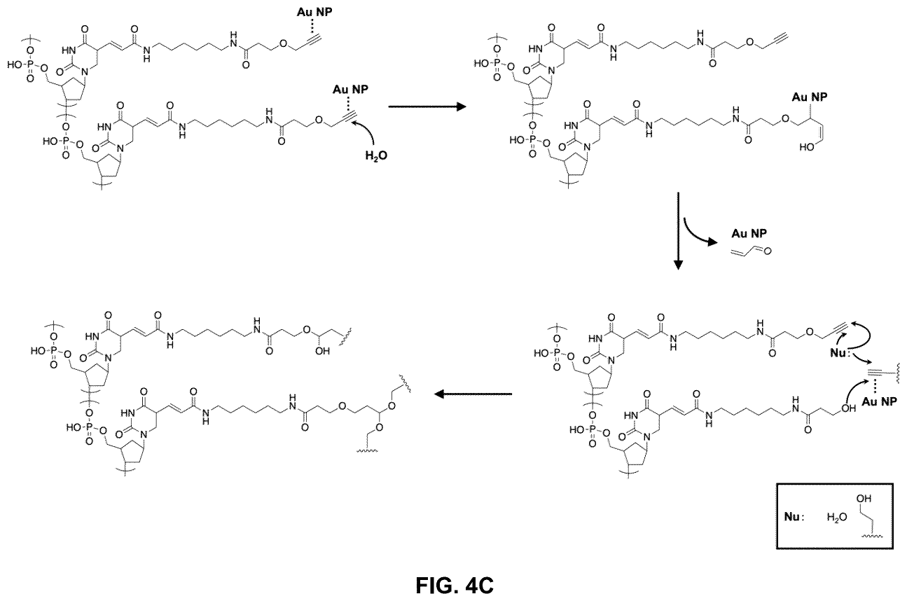

Hollow broad-spectrum nanozymes were synthesized in four steps (FIG. 4A). The general procedures of making hollow spherical nucleic acid particles were adapted from literatures (Choi, C, et al. Proc Natl Acad Sci USA. 2013 110 (19): 7625-7630; Cutler, J. I, et al. J Am Chem Soc 2011, 133 (24): 9254-7).

In the first step, Au nanoparticles were modified with multi-alkylthiol-terminated and propargyl-ether-modified RNase via gold/thiol linking chemistry. In the second step, Au-RNase complexes were further functionalized with multi-alkylthiol-terminated and propargyl-ether-modified protective DNA. Then, the resulting particles were further capped with structural supporters (alkylthiol-terminated and propargyl-ether-modified poly-thymine (T) sequences). Please note that co-functionalization with structural supporter oligonucleotides is important for the maintenance of structural integrity of hollow nanozymes. In the third step, the resultant nanoparticles from previous step were isolated via centrifugation and then were re-dispersed in phosphate-buffered saline (0.15 M) and then are incubated at the room temperature for 12 hours. Cross-linking takes place between the propargyl groups on the surface of the gold nanoparticles and along the modified T bases, yielding a densely packed, cross-linked DNA shell on the surface of Au nanoparticles. In the fourth step, Au nanoparticle cores were removed with an aqueous KCN solution (0.05 M), and the resulting hollow nanostructures is purified through multiple ultracentrifugations or extensive dialysis. The obtained hollow nanozyme is characterized with TEM and dynamic light scattering. Their enzymatic activity and RI-resistance is characterized using methods identical as those for broad-spectrum nanozyme with Au core.

Preparation and Characterization of Hollow Broad-Spectrum Nanozymes with Porous Shell

Hollow broad-spectrum nanozymes with porous shell were synthesized in five steps (FIG. 5A). The general procedures were similar as those of hollow broad-spectrum nanozymes without pores, with the difference that Au nanoparticles were co-functionalized with multi-alkylthiol-terminated and propargyl-ether-modified RNase/DNA, and specific protein molecules (e. g., bovine serum albumin naturally possessing a free thiol) which serves as “pore-making agent”. These protein molecules bind with Au nanoparticles via Au-thiol interactions, resulting in areas without polymerizable moieties on Au nanoparticle surface, leading to the formation of hollow nanozymes with porous shell after the removal of Au core. The pore size is tailorable by the size of the pore-making protein. In addition, we will study capacity of the porous hollow broad-spectrum nanozymes for loading small molecule drugs, such as anti-cancer, anti-virus, and anti-bacterial drugs (FIG. 5B).

### In Vitro Evaluation of Broad-Spectrum Nanozyme's Activities

Broad-spectrum nanozymes' were evaluated for intracellular potency, including cellular uptake, cytotoxicity and intracellular endogenous as well as exogenous RNA degradation. The cellular uptake of broad spectrum nanozyme among various cell lines were evaluated by dissolving cells along with the intracellular broad spectrum nanozymes using aqua regia, followed by Au concentration quantification using inductive-coupled-plasma atomic emission spectroscopy (ICP-AES). Cytotoxicity of broad-spectrum nanozyme was evaluated using cell viability measurement and the IC₅₀ of nanozyme is obtained by fitting the cell viability data into the four parameter logistic model (Yu, L, et al. Molecules 2019 24 (21)) using Origin software. Broad-spectrum nanozymes' degradation effect on endogenous intracellular RNA were evaluated by total cellular RNA extraction and gel electrophoresis. Liver cancer cells harboring HCV RNA replicon were used as a model system for the evaluation of broad-spectrum nanozymes' degradation effect on exogenous intracellular RNA. Quantitative real-time polymerase chain reaction (qRT-PCR) was used for HCV mRNA level quantification.

### Results

Expression and Purification of P19C/G89C Mutant RNase 1 (Pro19/Gly 89 Mutated into Cys, Respectively), as Well as A19C/G88C Mutant RNase A (Ala19/Gly 88 Mutated into Cys, Respectively)

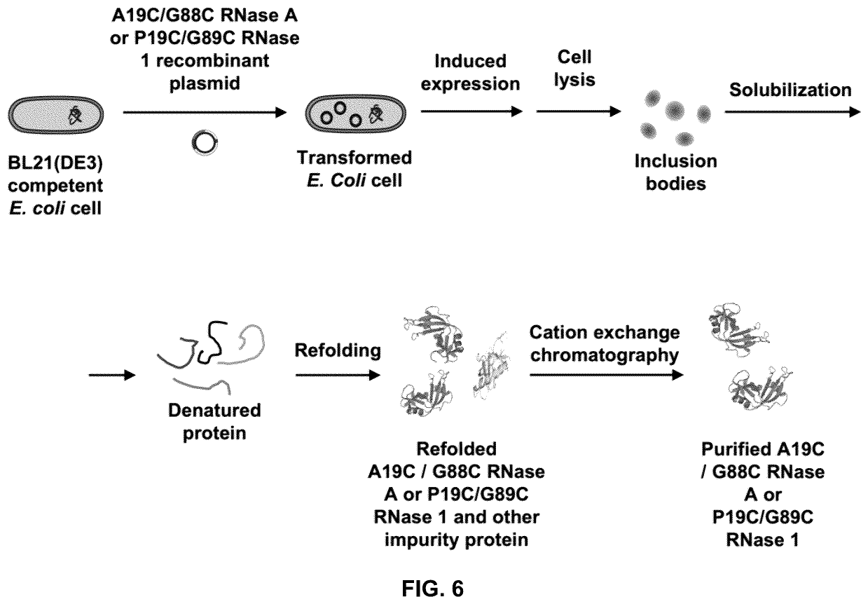

Mutant RNase A and RNase 1 are expressed and purified following reported procedures (FIG. 6) (Rutkoski, T, et al. Cancer Biology & Therapy 2011 12 (3): 208-214; Delcardayre, S, et al. Protein Engineering 1995 8 (3): 261-273). Specifically, based on protocols from cell suppliers, pET22b(+)/pET27b(+) plasmids encoding mutated RNase A or RNase 1 were transformed into BL21 (DE3) Competent E. coli with T7 RNA polimerase gene (New England BioLabs, strain genotype: fhuA2 [lon]ompT gal (λDE3) [dcm] ΔhsdS λ, DE3=A sBamHlo ΔEcoRI-B int:(lacl::PlacUV5:T7 gene1) i21 Anin5) and cultured in Lysogeny broth containing ampicillin overnight, followed by tiny amount of E. coli cells being transferred onto ampicillin-containing Lysogeny broth agar plate. After 8 h growth, a single colony was carefully picked and expanded by culturing in Lysogeny broth. E. coli cells were harvested after 12 h growth, and plasmids were extracted. The extracted plasmids were sent out sequenced to confirm the correctness of corresponding mutant RNase. For A19C/G88C mutant RNase A's and P19C/G89C RNase 1's large scale expression, 20 mL of overnight cultured corresponding E. coli cells were used to inoculate a large culture of 1.0 L terrific broth. After 4.5 h growth, isopropyl β-D-thiogalactoside was applied to induce the expression of mutant RNase A or RNase 1. E. coli cells were harvested after 3.0 h growth by centrifuge at 5000 rpm for 10 min.

The pelleted E. coli cells were first lysed using Bugbuster buffer (Millipore Sigma) following manufacturer's instructions and the inclusion bodies were collected by centrifugation. The obtained inclusion bodies were further washed for 3 more times with PBS before solubilized by solubilization buffer (Tris-HCl PH 7.80, ethylenediaminetetraacetic acid 1.00 mM, NaCl 0.400 M, urea 6.00 M, DTT 100 mM). The fully dissolved inclusion bodies solution was centrifuged to remove any insoluble precipitate, followed with dilution by 10-fold applying degassed 20 mM acetic acid solution. The diluted solution of inclusion bodies was dialyzed (ThermoFisher Scientific, 10 kDa molecular weight cut-off) overnight against 20 mM acetic acid (pre-purged with nitrogen) to obtained denatured mutant RNase solution.

For preparing enzymatically-active protein, refolding was carried out for the denatured mutant RNase A or RNase 1. Specifically, the solution containing denatured protein was slowly added into refolding solution (nitrogen pre-purged 0.10 M pH 8.0 Tris-HCl buffer, with ethylenediaminetetraacetic acid 10 mM, NaCl 0.10 M, reduced glutathione 1.0 mM, oxidized glutathione 0.20 mM, L-arginine 0.50 M). The solution was kept under 4° C. for over 5 days to facilitate the refolding of denatured RNase.

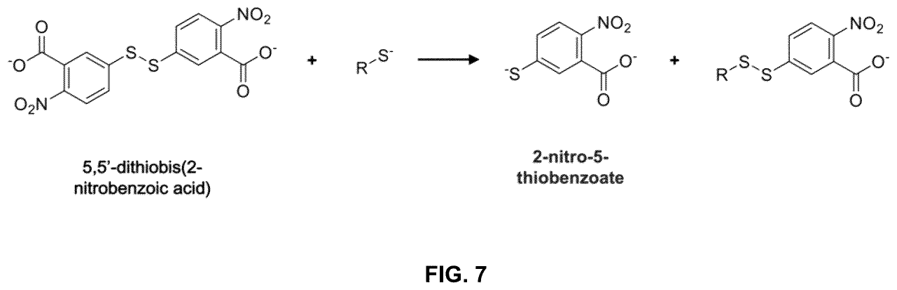

After refolding process, the refolded protein was concentrated applying centrifugal filters (10 kDa molecular weight cut-off, Millipore Sigma). For protecting the thiol group from oxidization, 5,5′-dithiobis(2-nitrobenzoic acid) (10-fold excess) was applied to cap the thiol group of RNase in Tris-HCl buffer (pH 8.0) containing 10 mM ethylenediaminetetraacetic acid (FIG. 7). The excess 5,5′-dithiobis(2-nitrobenzoic acid) was removed applying NAP-10 desalting column (GE healthcare) following manufacturer's protocols. Finally, the thiol-capped mutant RNase A or RNase 1 was purified using a HiTrap SP cation-exchange column (G E healthcare) and eluted with a linear gradient of NaCl (0.15-1.0 M) in 50 mM pH 5. ONaOAc buffer. FIG. 8 showed the typical fast protein liquid chromatography data of A19C/G88C RNase A. Upon usage, 1.0 mM dithiothreitol treatment was applied to de-cap the protected thiol and RNase was desalted into proper buffer using NAP-5 desalting column (GE Healthcare).

Tagging of P19C/G89C/A19C/G88C with Lipoic Acid Moiety (LA-P19C/G89C/A19C/G88C) Via Click Chemistry

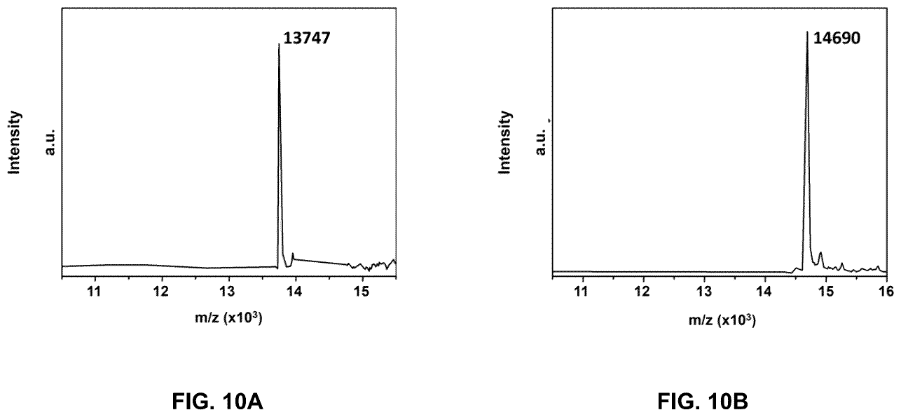

LA-A19C/G88C/P19C/G89C was constructed via two steps of reactions (FIG. 8). First, A19C/G88C mutant RNase A or P19C/G89C RNase 1 was reacted with 10-fold molar excess of dibenzylcyclooctyne-PEG₄-maleimide to introduce the clickable dibenzylcyclooctyne functional groups onto mutated cysteine site. The reaction was carried out in 0.10 M pH 7.0 phosphate buffer containing 10 mM ethylenediaminetetraacetic acid for 3 hours. The excess dibenzylcyclooctyne-PEG₄-maleimide was removed with NAP-5 desalting column. Next, the obtained dibenzylcyclooctyne-containing P19C/G89C RNase 1 or A19C/G88C mutant RNase A was further reacted with lipoamido-PEG₃-azide (10-fold molar excess) in PBS for 5 hours to yield LA-P19C/G89C/A19C/G88C. Fast protein liquid chromatography was applied for final purification using a HiTrap SP cation-exchange column and eluted with a linear gradient of NaCl (0.15-1.0 M) in 50 mM pH 5.0 NaOAc buffer. The purity of LA-P19C/G89C/A19C/G88C was checked by MALDI-TOF (FIG. 10).

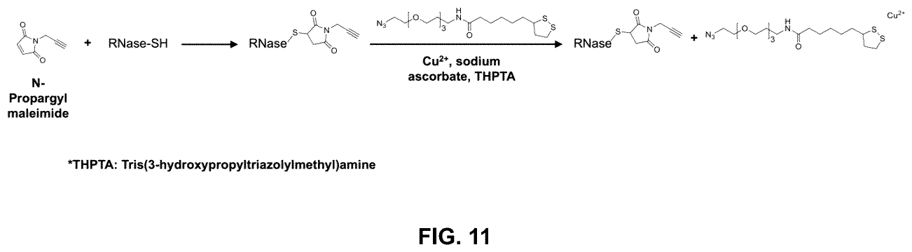

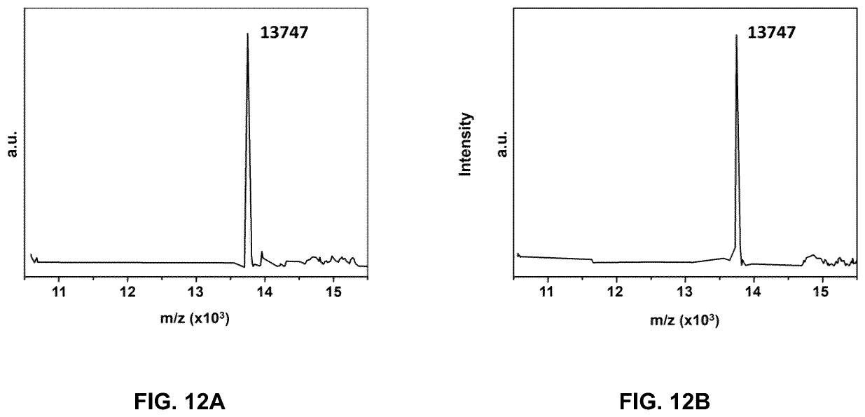

It should be mentioned that classical copper assisted click chemistry was also used to tag cysteine-containing mutant RNase with lipoic acid moiety (FIG. 11). However, the reaction exhibited extremely low yield probably because of the strong chelating interaction between copper and thiol, and no product was detected in MALDI-TOF (FIG. 12).

### Enzymatic Activity Evaluation of P19C/G89C/A19C/G88C and LA-P19C/G89C/A19C/G88C

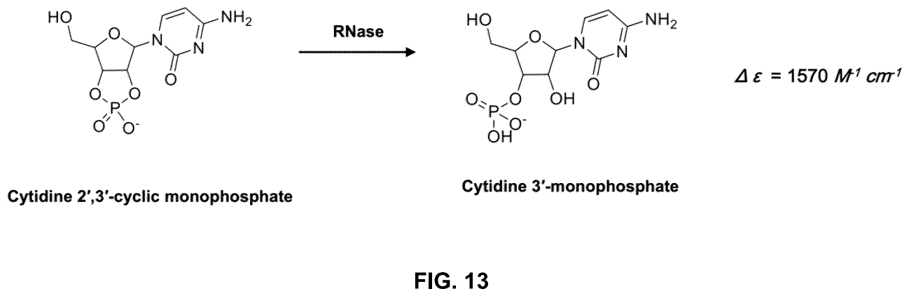

The RNase's ribonucleolytic activity is of great significance for the gene silencing effect of nanozyme. Thus, following a method developed by Crook et al. (FIG. 13) (Crook, E, et al. Biochemical Journal 1960 74:234-238), the steady-state kinetic parameters of P19C/G89C RNase 1 and A19C/G88C mutant RNase A, as wells as LA-P19C/G89C and LA-A19C/G88C were characterized and compared with those of wild type RNase 1 and RNase A. Specifically, cytidine 2′,3′-cyclic monophosphate (substrate) of different concentrations was mixed with 1.00 μM of RNase and the mixtures' time-dependent absorbance at 286 nm was recorded. As cytidine 2′,3′-cyclic monophosphate being hydrolyzed into cytidine 3′-monophosphate with higher extinction coefficient, the absorbance at 286 nm keeps increasing. Based on the data of absorbance increasing, the reaction's initial velocity was calculated and fitted with Lineweaver-Burk plot to determine the RNase's steady-state kinetic parameters.

Compared with wild-type RNase 1 and RNase A, only slightly decreased activity was observed for either P19C and G89C or A19C and G88C (Table 1), which is in consistence with the previous reported data (Rutkoski, T, et al. Cancer Biology & Therapy 2011 12 (3): 208-214). Specifically, after tagging with lipoic acid moiety, the K_{cat}/K_{M} of LA-P19C/G89C/A19C/G88C was found to be about 90% of those for P19C/G89C/A19C/G88 without tag, suggesting that lipoic acid moiety tagging did not result in a significant change of RNase's catalytic activity.

TABLE 1
Steady-state kinetic parameters of wild-type
RNase A, RNase 1, A19C, G88C, P19C, G89C,
LA-A19C, LA-G88C, LA-P19C and LA-G89C.

| | k_{cat}/K_{M} (10² M⁻¹ × s⁻¹) |
|---|---|
| Wild-type RNase A | 9.75 ± 0.446 |
| Wild-type RNase 1 | 44.2 ± 0.262 |
| A19C | 7.58 ± 0.135 |
| G88C | 7.28 ± 0.415 |
| P19C | 32.5 ± 0.855 |
| G89C | 31.7 ± 0.964 |
| LA-A19C | 6.82 ± 0.402 |
| LA-G88C | 6.42 ± 0.377 |
| LA-P19C | 29.3 ± 0.823 |
| LA-G89C | 28.3 ± 0.717 |

### RNase Loading Conditions Optimization

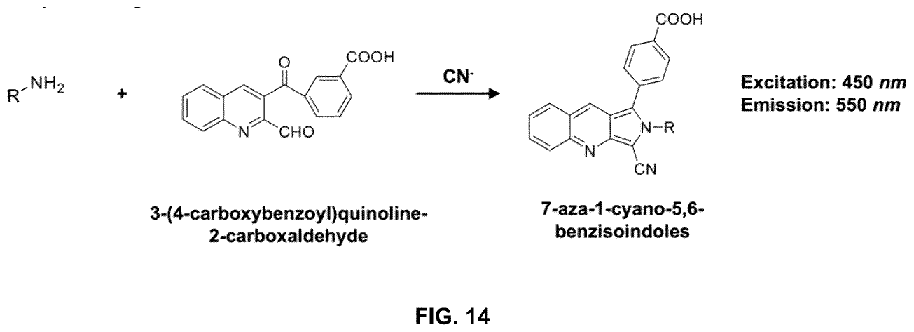

Even with the help of tagged lipoic acid moiety, there is still possibility that the modified mutant RNase may nonspecifically bind onto Au NPs (NPs). For optimizing the RNase binding ensuring that enzymes bind onto Au nanoparticle exclusively via gold-sulfur bond, the loading conditions of RNase on Au nanoparticles was studied. Considering the similarity in overall structures and amino acid sequences, wild type RNase A was applied as a model enzyme and different additive molecules effects on enzymes' nonspecific binding was evaluated. Three additive molecule candidates, p-toluenesulfonate, citrate, and tween 20 were chosen since they only weakly interact with Au NPs and have no interference on the binding of thiol-containing ligands. For the quantification of the amount of RNase A on Au NPs, the Au core of Au-RNase A complex was dissolved with KCN (0.05 M) solution for 1 h and the released RNase A were quantified with CBQCA Protein Quantitation Kit (ThermoFisher Scientific) following manufacturer's protocols. This kit characterize the protein concentration according to the fluorescence signal generated from the reaction between the primary amine of protein molecules and 3-(4-carboxybenzoyl) quinoline-2-carboxaldehyde (FIG. 14).

As shown in Table 2, None of the three additive molecules alone totally suppressed the nonspecific binding of wild type RNase A onto Au NPs. For p-toluenesulfonate, as the concentration gradually increased to 100 mM, the number of wild type RNase A nonspecifically bound on one Au NP gradually decreased from 16.3 to 8.01 (Table 2). With further increased p-toluenesulfonate concentration up to 200 mM, the number of wild type RNase A bound on Au NP remains to be 8.33. As for citrate, the number of RNase A bound on one Au NP decreased from 15.9 to 7.30 with citrate concentration increased to 50.0 mM. Further elevating citrate concentration up to 200 mM did not lead to less nonspecific binding, instead, it promoted more RNase A (13.9 per one Au nanoparticle) nonspecifically bind onto Au NPa (Table 2). Tween 20 outperformed p-toluenesulfonate and citrate. At concentration of 1.25%, the number of RNase A per Au NP was reduced to 2.00 (Table 3), but still not reaching zero. By combining 50.0 mM citrate with 1.25% tween 20, zero RNase A loading was detected binding on Au NPs, suggesting the nearly complete suppression of RNase A's nonspecific binding. Accordingly, 1.25% tween 20 with 50.0 mM citrate was selected as the conditions for the loading of lipoic acid tagged mutant RNase 1 or RNase A onto Au NPs.

TABLE 2
Loading number of wild type RNase A per single Au NP
with different concentration of citrate or p-toluenesulfonate
as additives (with 0.0100% tween 20).

| | | Number of RNase A/Au NP | |
|---|---|---|---|
| Additive concentration | | p-toluenesulfonate as additive | Citrate as additive |
| 0.00 | mM | 16.3 ± 1.03 | 15.9 ± 0.790 |
| 10.0 | mM | 10.7 ± 0.946 | 11.7 ± 0.310 |
| 25.0 | mM | 10.4 ± 0.635 | 7.40 ± 0.725 |
| 50.0 | mM | 8.94 ± 0.938 | 7.30 ± 0.554 |
| 100 | mM | 8.01 ± 1.11 | 8.98 ± 0.420 |
| 150 | mM | 7.95 ± 0.836 | 10.1 ± 0.885 |
| 200 | mM | 8.33 ± 0.735 | 13.9 ± 0.883 |

TABLE 3
Loading number of wild-type RNase A per single Au NP with
different concentration of tween 20 as additives.

Number of RNase A/Au NP

| Concentration of | 0.00 mM citrate | 50.0 mM citrate |
|---|---|---|
| Tween 20 | as additive | as additive |
| 0.00200% | 38.0 ± 0.637 | 15.2 ± 0.637 |
| 0.0100% | 14.7 ± 1.08 | 7.31 ± 0.550 |
| 0.0250% | 11.6 ± 0.872 | 2.97 ± 1.14 |
| 0.0500% | 11.8 ± 0.165 | 2.81 ± 1.08 |
| 0.250% | 9.66 ± 0.623 | 2.18 ± 0.942 |
| 1.25% | 1.95 ± 1.03 | 0.00 ± 0.729 |

Demonstration of the RNase Inhibitor Resistance of Broad-Spectrum Nanozyme and Hollow Broad-Spectrum Nanozyme with Varied RNase and Poly-Deoxyadenosine (A6) Loading Amount

Because of the coverage from the densely packed oligonucleotides, the RNase A in nanozyme was protected from being accessible by RNA not complimentary to the probe DNA. Such cooperativity leads to nanozymes' selective degradation toward target RNA.

Mammalian RNase inhibitor (RI) is a type of acidic protein specifically binds and inactivates pancreatic type RNase, including RNase A (Kobe, B, et al. Journal of Molecular Biology 1996 264 (5): 1028-1043). Considering the steric hindrance and RI's poly anionic nature similar to non-complimentary control RNA segments, the nanozyme was expected to be RI resistant. For demonstration, broad-spectrum nanozyme and hollow broad-spectrum nanozyme with protecting DNA composed of a poly-deoxyadenosine (A₆) with a PEG spacer and various RNase loading were evaluated for their RI-resistance.

For maximized RNase activity, the loading density of LA-A19C is expected to be at its extreme. However, with such high coverage of LA-A19C on Au NPs, the loading density of poly A₆ is expected to be significantly lowered, which may result in nanozymes' poor RI-resistance. Without RI-resistance, the nanozyme would readily be inactivated by RI in cytosol, thus losing its cytotoxicity.

Accordingly, in order to find the optimized conditions of both high RNase activity and RI-resistance, nanozymes with varied LA-A19C loading density was prepared and studied. The loading density of both LA-A19C and poly A₆ were quantified and summarized in Table 4. With increased amount of LA-A19C functionalized on Au NPs, the loading density of poly A₆ decreased. Surprisingly, even with nearly saturated LA-A19C loading density of about 53 RNase A per Au NP, there was still averagely about 54 poly A₆ co-functionalized.

TABLE 4
Average number of LA-A19C and poly A6 molecules per Au NP.

| | Number of LA-A19C/Au NP | Number of poly A₆/Au NP |
|---|---|---|
| Au-poly A₆ | / | 144.2 ± 1.2 |
| Nanozyme1 | 12.4 ± 2.6 | 109.8 ± 1.6 |
| Nanozyme2 | 24.8 ± 3.3 | 108.5 ± 1.1 |
| Nanozyme3 | 28.8 ± 1.1 | 106.2 ± 2.3 |
| Nanozyme4 | 36.5 ± 2.3 | 99.7 ± 2.7 |
| Nanozyme5 | 44.5 ± 3.0 | 74.9 ± 2.1 |
| Nanozyme6 | 53.4 ± 2.9 | 54.1 ± 1.9 |

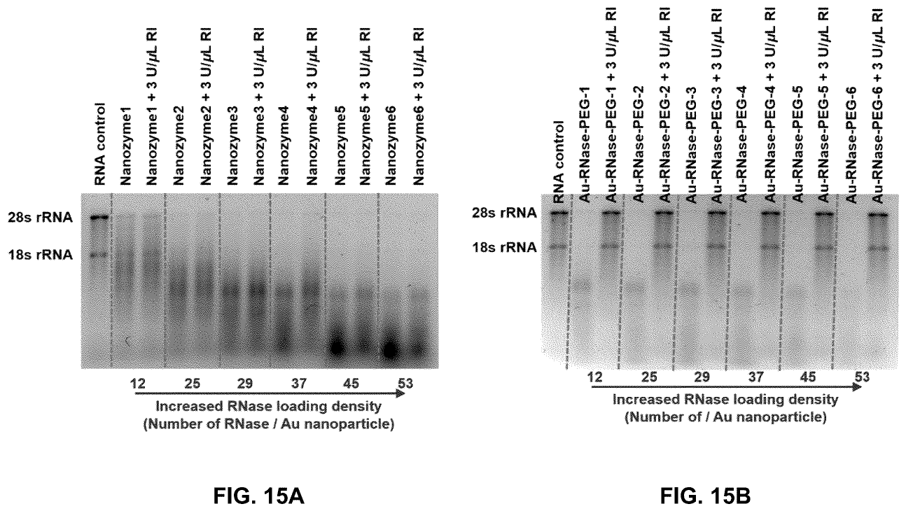

All prepared nanozymes were evaluated for their RNase activity and RI-resistance using total cellular RNA as substrates. For comparison, the corresponding Au-LA-A19C-PEG500 NPs with same LA-A19C loading density but without poly A₆ were also evaluated. As shown in FIG. 15, from nanozyme 1 to 6, the RNase activity increased, which was consistent with the trend of increased number of RNase per Au NP. Moreover, at 3 U/UL RI (1 U is defined as the amount of RI required to inhibit the activity of 5 ng of RNase A by 50%, measured by the inhibition of hydrolysis of cytidine 2,3′-cyclic monophosphate), all nanozyme still showed strong degradation towards total cellular RNA, suggesting that they all possessed decent RI-resistance. However, it was also noticed that starting from nanozyme4, slightly decreased RNase activity was observed with the existence of RI, suggesting that with gradually increased LA-A19C and decreased poly A₆ loading density, nanozymes' RI-resistance indeed decreased. On the other hand, all Au-LA-A19C-PEG500 NPs without poly A₆ lost most of their activity with 3 U/μL RI, implying their poor RI-resistance. These results demonstrated that using short oligonucleotide (poly A₆), the prepared nanozymes with non-selective RNase activity maintained their RI-resistance.

Demonstration of Broad-Spectrum Nanozymes' Cytotoxicity to A549 Cell Line (Lung Cancer) (Nanozyme with Different Loading Density of RNase and DNA)

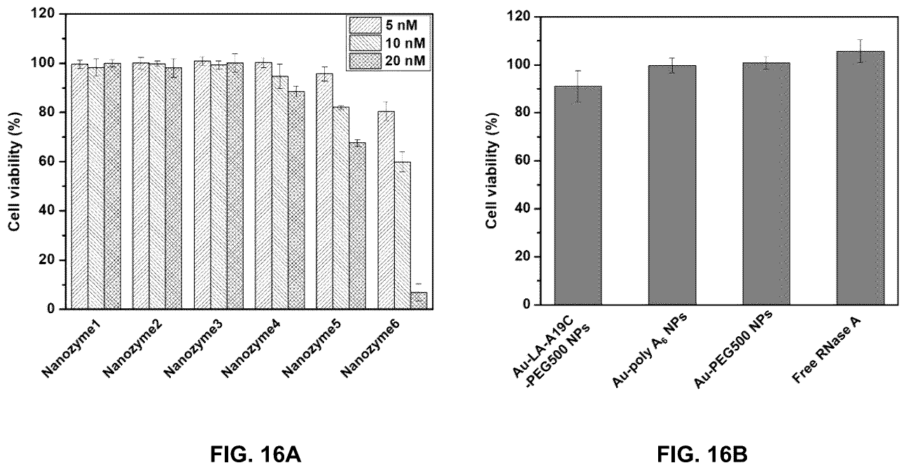

The cytotoxicity of broad-spectrum nanozyme with protecting DNA composed of an A₆ strand with a PEG-spacer and various RNase loading density was first evaluated with A549 cells. For comparison, the cytotoxicity of corresponding Au-RNase-PEG NPs, Au nanoparticles capped with A₆ strands with a PEG spacer, Au-PEG NPs, and free RNase were also evaluated. As shown in FIG. 16A, within 5 nM to 20 nM concentration range, nanozyme1, 2 and 3 showed no obvious influence on cell viability after 48 h treatment, suggesting their low cytotoxicity. With gradually elevated RNase loading density, nanozyme4, 5 and 6 presented increased cytotoxicity to A549 cells. Specifically, after treatment of 20 nM for 48 h, the viability of cells treated by above three types of nanozymes were 88.5%, 67.6%, and 6.91%. These results proved the cytotoxicity of broad-spectrum nanozyme to A549 cell line.

On the other hand, as shown in FIG. 16B, without protecting DNA, Au-RNase-PEG₅₀₀ NPs possessing no RI-resistance showed very little effect on cell viability. Moreover, cell treated with free RNase, Au-poly A₆ NPs without RNase, and Au-PEG nanoparticles also exhibited almost no decrease in cell viability. These results suggested that the broad-spectrum nanozymes' structure of scaffold co-functionalized with both acting and protecting moieties is crucial for their cytotoxicity.

Study of the Cellular Uptake of Broad-Spectrum Nanozyme with Different Loading Density of RNase and DNA

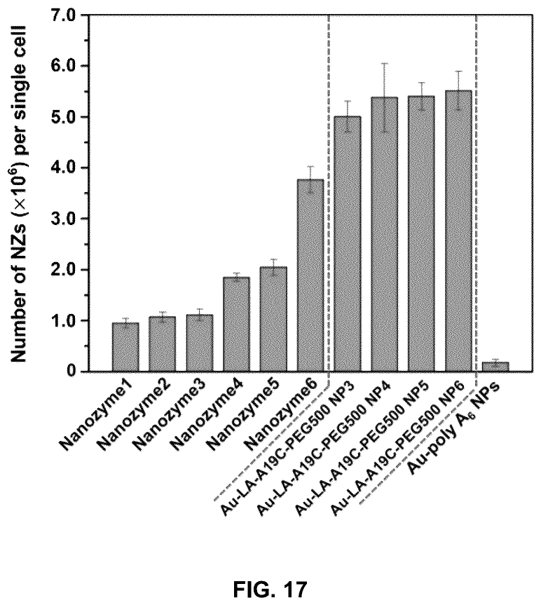

The cellular uptake of broad-spectrum nanozyme to A549 cells was studied by ICP-AES. As shown in FIG. 17, the cellular uptake for Au-poly A₆ NPs without RNase was found to be about averagely 1700 NPs per cell. This was consistent with the reported data about the cellular uptake of spherical nucleic acid (Choi, C, et al. Proc Natl Acad Sci USA. 2013 110 (19): 7625-7630). Meanwhile, the endocytosis for Au-RNase-PEG NPs without DNA was much higher than that of Au-poly A₆ NPs, which was found to be about 30 times higher than the cellular uptake of Au-poly A₆ NPs without RNase. However, as discussed in previous section, Au-RNase-PEG NPs did not present obvious cytotoxicity, implying that that Au NPs merely functionalized with RNase was not enough to induce obvious cytotoxicity. The cellular uptake of broad-spectrum nanozyme was between those of Au-poly A₆ and Au-RNase-PEG₅₀₀ NPs. When the RNase loading density was low, the nanozyme1, nanozyme2 and nanozyme3 exhibited a cellular uptake of about 1,000,000 NPs per cell. This was far less than the cellular uptake of Au-RNase-PEG NPs, but much higher than that of Au-poly A₆ NPs. With increased RNase loading density, higher cellular uptake was observed and averagely around 2,000,000 NPs per cell were found for nanozyme4 and nanozyme5. As reported in previous section, Nanozyme4 and nanozyme5 started to show some cytotoxicity as decreased cell viability (89% and 67% cells viable with 20 nM nanozyme4 and nanozyme5 treated for 48 h). With further increased loading density of RNase, cellular uptake of 3,800,000 NPs per cell were found for nanozyme6. This was over two times increased as compared with nanozyme4 and nanozyme5. Moreover, A549 cells treated by nanozyme6 showed drastically decreased cell viability (˜7% viable with 20 nM nanozyme6 treated for 48 h).

Thus, based on above results, it was concluded that the cytotoxicity of broad-spectrum nanozyme depends on both RI-resistance and cellular uptake efficiency. Without RI-resistance, even with highly efficient endocytosis, no obvious cytotoxicity would be observed. Meanwhile, for nanozyme possessing strong RI-resistance, they still did not show obvious cytotoxicity without efficient cellular uptake. Only strong RI-resistance and efficient cellular uptake together resulted in high cytotoxicity.

### Evaluation of the Effects of DNA Length on Nanozymes' Activity, RI-Resistance and Cytotoxicity.

Besides the LA-A19C loading density, the length of DNA (i.e., the number of nucleotides, herein) was expected to be another important parameter affecting the cytotoxicity of nanozyme. Accordingly, besides poly A₆, nanozyme were prepared with two different length anti-HCV oligonucleotides, 18-bases DNA1 (5′-CTT-GAA-TGT-AGA-GAT-GCG-PPP-SH-3′, SEQ ID NO:7) and 39-bases DNA2 (5′-TTT-TGG-TTT-TTC-TTT-GAG-GTT-TAG-GAT-TTG-TGC-TCA-TGG-PPP-SH-3′, SEQ ID NO:8), using Au-LA-A19C NPs of 53 LA-A19C per Au NP. As shown in Table 5, nanozymes prepared from poly A₆ had higher oligonucleotide loading density (54 poly A₆ per Au NP) than nanozymes prepared from DNA1 and DNA2 (43 and 40 oligonucleotides per Au NP, respectively). This is probably due to the higher electrostatic repulsion between adjacent oligonucleotides of longer bases.

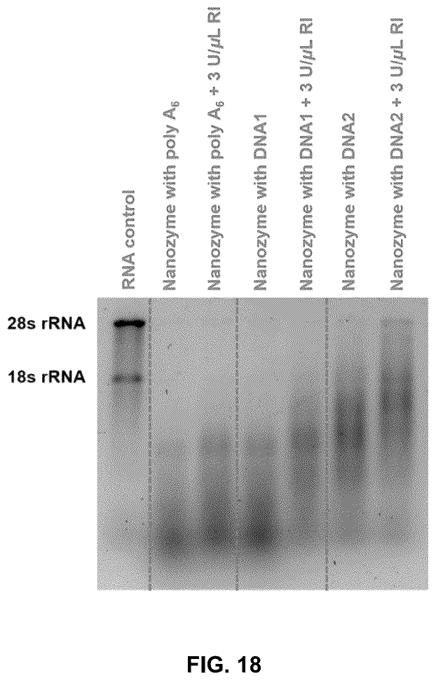

Next, the RNase activity and RI-resistance of prepared nanozymes were evaluated. As shown in FIG. 18, all three types of nanozymes presented strong degradation effects on total cellular RNA. Compared with nanozyme of poly A₆, those of 18-bases DNA1 and 39-bases DNA2 exhibited obviously decreased RNase activity. This is probably due to the stronger electrostatic repulsion between longer oligonucleotides on Au NPs and RNA substrates. Such electrostatic repulsion resulted in less efficient binding of RNA substrate with LA-A19C, thus leading to lowered RNase activity. On the other hand, no significant difference in RI-resistance was observed for three types of nanozymes, as all three of them presented slightly decreased RNase activity at 3 U/μL RI.

TABLE 5
Average number of oligonucleotides for nanozymes
prepared with DNA of varied length.

| | Average number |
|---|---|
| | of DNA/Au NP |
| Nanozyme with poly A₆ | 54.1 ± 1.9 |
| Nanozyme with 18 bases anti-HCV DNA1 | 42.5 ± 1.8 |
| Nanozyme with 39 bases anti-HCV DNA2 | 40.3 ± 2.6 |

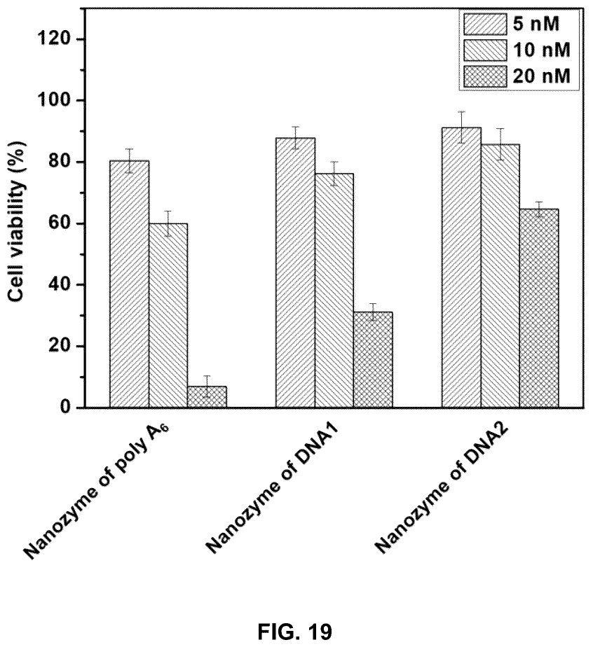

The cytotoxicity of three types of nanozymes were evaluated using A549 cells. As shown in FIG. 19, with increased oligonucleotide length, the corresponding nanozyme presented lower cytotoxicity. Specifically, after treated with 20 nM for 48 h, about 7% viability was observed for cells treated with nanozyme of poly A₆. Meanwhile, cells treated with nanozyme of DNA1 (18 bases) and DNA2 (39 bases) showed significantly higher viabilities of about 31% and 65%, implying the corresponding nanozymes' lower cytotoxicity.

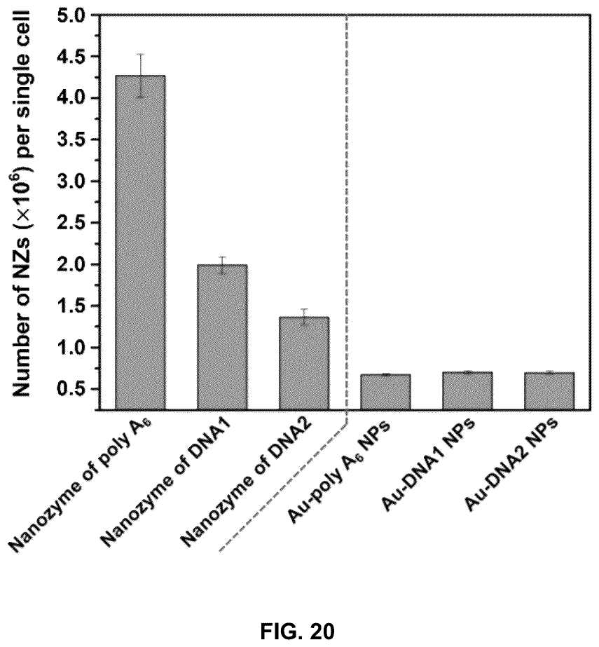

For investigating the possible reasons behind such drastic difference of cytotoxicity, the endocytosis of three types of nanozymes with difference oligonucleotide length was studied. As shown in FIG. 20, with increased length of oligonucleotides, the cellular uptake of corresponding nanozyme decreased significantly. Specifically, treated with nanozyme of poly A₆, endocytosis of averagely about 3,800,000 NPs per cell were found. With longer oligonucleotides, the cellular uptake for nanozymes of DNA1 and DNA2 dropped to about 1,500,000 and 900,000 NPs per cell. Thus, the lowered cytotoxicity for nanozyme with increased length of oligonucleotides was likely to be the result of decreased endocytosis efficiency. This trend was also consistent with the results of last section that less cellular uptake for nanozymes was accompanied by weaker cytotoxicity.

From above results, it was concluded that the cytotoxicity of nanozyme was correlated with both its RI-resistance and endocytosis efficiency. Deficiency of either one would result in nanozyme's low cytotoxicity.

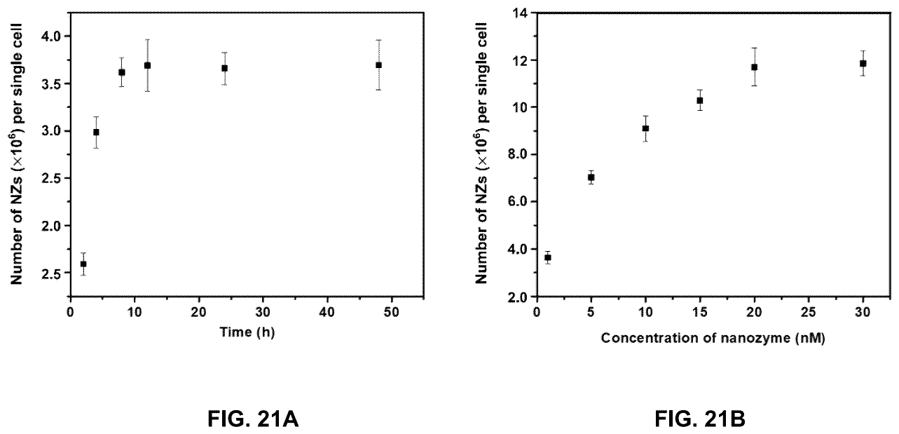

First, the time-dependence of endocytosis was evaluated with ICP-AES, as shown in FIG. 21A, at 1 nM concentration, the amount of Au NPs per cell kept increasing within the first 12 h of incubation, then became plateaued after 12 h till 48 h, suggesting the saturation of endocytosis after 12 h incubation. Meanwhile, as shown in FIG. 21B, at fixed 12 h incubation time, the cellular uptake for nanozyme increased with elevated nanozyme concentration from 1 nM to 20 nM. Further increase the concentration of nanozyme to 30 nM did not result in an obvious elevation in cellular uptake.

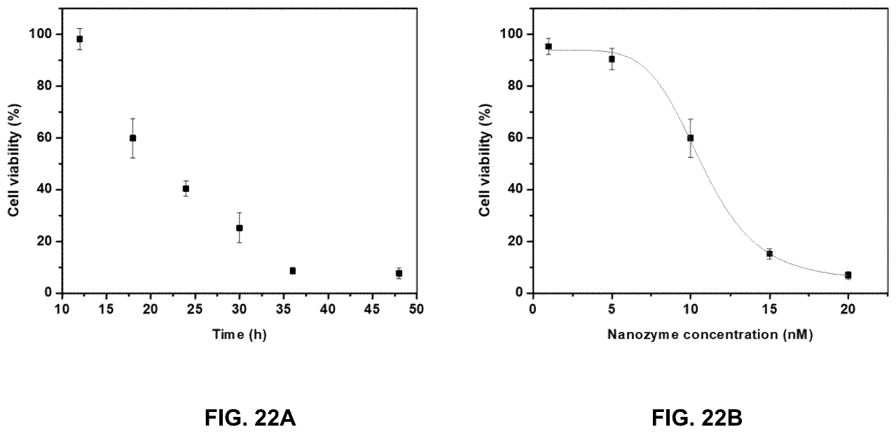

Next, the time-dependent cytotoxicity of nanozyme was evaluated. As shown in FIG. 22A, at 20 nM of nanozyme and 12 h incubation, the cell viability was found to be close to 100%, suggesting that before 12 h, the majority of cells remained alive. After 12 h till 36 h, the cell viability gradually decreased with longer incubation time. At 36 h incubation, only about 8% cell viability was detected, and further prolonging incubation till 48 h did not result in lower cell viability. From nanozyme's time-dependent endocytosis study in previous section, it was found that most cellular uptake of nanozyme happened within the first 12 h of incubation. Thus, there appeared to be a lag time of 12 h for nanozymes' cytotoxicity to be reflected as decreased cell viability. Within the first 12 h of incubation, nanozymes were continuously taken into cells without causing significant cell death. After 12 h, the cytotoxicity of nanozyme started to reveal as dropped cell viability.

Meanwhile, the nanozymes' concentration-dependent cytotoxicity was also evaluated. As shown in FIG. 22B, after 48 h of incubation, the cell viability decreased with higher concentration of nanozyme. By fitting the cell viability data with four parameter logistic model (Yu, L, et al. Molecules 2019 24 (21)), the IC₅₀ of nanozyme to A549 cells was determined to be 10.7 nM. This is almost two orders of magnitudes lower than the IC₅₀ of reported natural or mutant cytotoxic RNase on A549 cells under similar conditions (Hoang, T, et al. Molecular Cancer Therapeutics 2018 17 (12): 2622-2632).

Intracellular RNA Analysis of A549 Cells Treated with Broad-Spectrum Nanozyme

To better understand the mechanism of nanozymes' cytotoxicity, the intracellular RNA of A549 cells treated with various concentrations of nanozyme over different length of time was analyzed with gel electrophoresis.

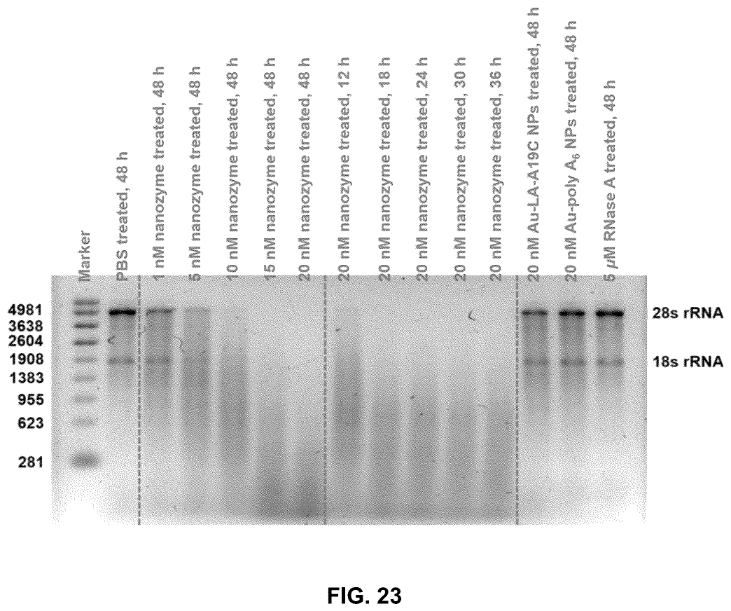

As shown in FIG. 23, compared with PBS-treated A549 cells, those treated with nanozyme exhibited obvious intracellular RNA degradation. Specifically, after treating with 1 nM nanozyme for 48 h, noticeable 18s and 28s rRNA degradation was observed. With elevated nanozyme's concentration to 5 and 10 nM, more 18s and 28s rRNA was degraded. When cells were treated with 15 and 20 nM of nanozyme, almost no 18s and 28s rRNA band could be observed and most of them were degraded into small pieces. Such increased degree of rRNA degradation was consistent with the results of cell viability measurement shown in FIG. 22B. With higher concertation of nanozyme treatment, lower cell viability was observed. Majority of the cells were found to be dead when nanozyme's concentration reached 15 nM. On the other hand, the intracellular 18s and 28s rRNA of cells treated with 20 nM Au-LA-A19C-PEG500 NPs, Au-poly A₆ NPs or 5 μM free RNase A did not present very obvious degradation, suggesting that no intracellular RNA degradation was induced. This was consistent with the previous results that no cytotoxicity was observed when cells were incubated with above control NPs.

Moreover, with fixed nanozyme concentration of 20 nM, serious 18s and 28s rRNA degradation was observed after 12 h of incubation. However, as shown in FIG. 22B, under this condition, about 98% cells were viable. Therefore, it is likely that the initial 12 h of incubation is a lag time for nanozyme's cytotoxicity, during which nanozymes were continuously taken into cells and the intracellular RNA degradation was initiated. But cells remained viable. With longer incubation time of 18 h till 48 h, almost no intact 18s or 28s rRNA could be observed (FIG. 22), and decreased cell viability was detected (FIG. 22B).

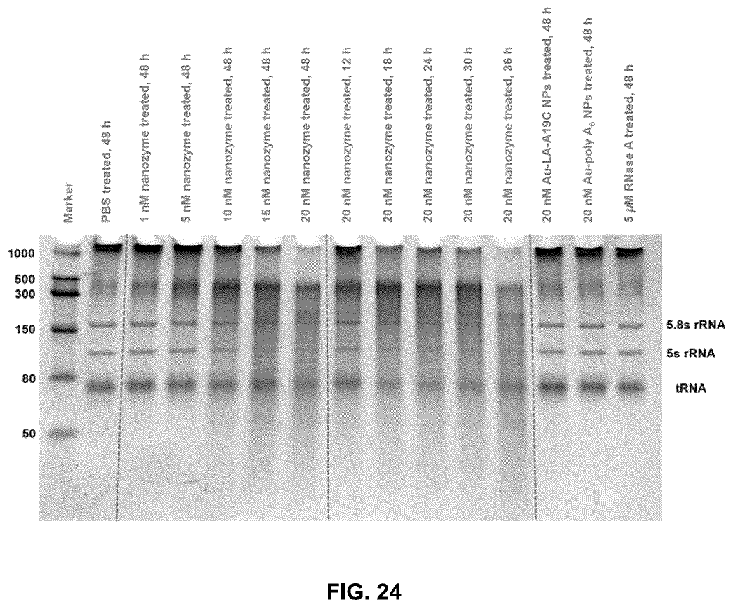

Not only large sized 18s and 28s rRNA, smaller sized 5s, 5.8s rRNA and tRNA of A549 cells treated with nanozyme was also analyzed with electrophoresis using TBE-urea gel. As shown in FIG. 24, after treating with higher concentration of nanozyme for 48 h, more serious 5s, 5.8s rRNA and tRNA degradation was observed. However, the degradation of 5s, 5.8s rRNA and tRNA was not as obvious as for 18s and 28s rRNA in FIG. 23, suggesting that nanozyme was more potent in degrading large sized intracellular RNA. Meanwhile, when nanozyme's concentration was fixed at 20 nM, after 12 h incubation, only very slight 5s, 5.8s rRNA and tRNA degradation was observed. Obvious degradation was found for cells with longer incubation from 18 h till 48 h. Overall, the intracellular degradation of 5s, 5.8s rRNA and tRNA was not as serious as 18s and 28s rRNA. Moreover, it should be mentioned that no obvious intracellular RNA degradation was observed for A549 cells treated with 20 nM Au-LA-A19C-PEG500 NPs, Au-poly A₆ NPs or 5 μM free RNase A.

### Evaluation of Broad-Spectrum Nanozymes' Cytotoxicity on HeLa, K562, Ramos and CCRF-CEM Cells

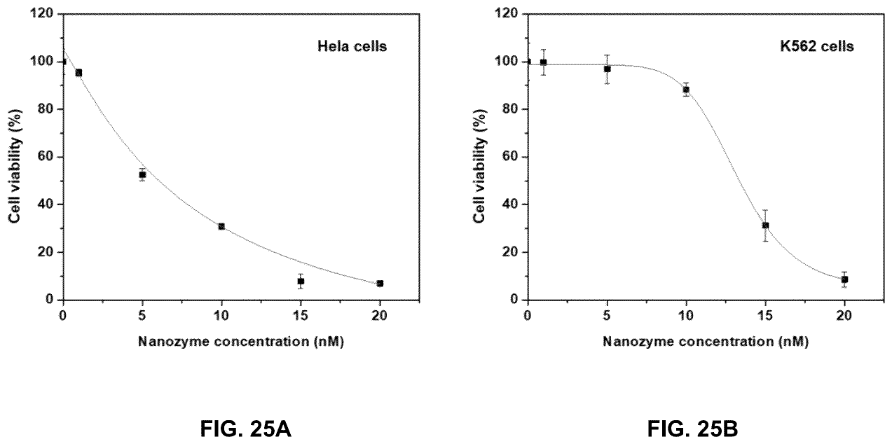

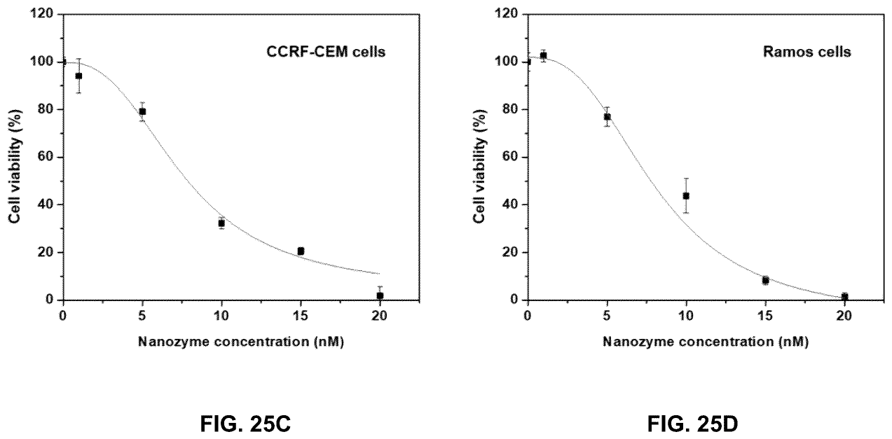

Besides A549 cells, the nanozymes' cytotoxicity was also evaluated with other cell lines, including HeLa, K562, CCRF-CEM, and Ramos cells. As shown in FIG. 25, all types of cells presented decreased cell viability with increased nanozyme concentration, suggesting that nanozyme's cytotoxicity was general for multiple types of tumor cell lines.

TABLE 6
IC₅₀ of nanozyme, Au-poly A6 NPs, Au-LA-A19C NPs
and free RNase A on various types of tumor cells.

| | A549 | | HeLa | | K562 | | CCRF-CEM | | Ramos | |
|---|---|---|---|---|---|---|---|---|---|---|
| Nanozyme | 10.7 | nM | 6.06 | nM | 13.3 | nM | 7.88 | nM | 7.68 | nM |
| Au-poly A6 NPs | >40.0 | nM | >40.0 | nM | >40.0 | nM | >40.0 | nM | >40.0 | nM |
| Au-LA-A19C NPs | 38.0 | nM | 10.1 | nM | 24.6 | nM | 17.6 | nM | 16.8 | nM |
| Free RNase A | >5.00 | μM | >5.00 | μM | >5.00 | μM | >5.00 | μM | >5.00 | μM |

By fitting the concentration-dependent cell viability into four parameter logistic model (Yu, L, et al. Molecules 2019 24 (21)), the IC₅₀ of nanozymes as well as Au-poly A₆ NPs without LA-A19C, Au-LA-A19C NPs without poly A₆ and free RNase A on various types of tumor cells were determined and summarized in Table 6. With only oligonucleotides, the Au-poly A₆ NPs (up to 40 nM in cell culture medium) did not show any effect on cell viability, proving their low cytotoxicity. This is consistent with the reported results for the spherical nucleic acids' high biocompatibility (Rosi, N, et al. Science 2006 312 (5776): 1027-1030). Meanwhile, within 5 μM, free RNase A did not present any influence on cell viability, indicating its extremely low cytotoxicity. This is also consistent with reported data from various publications (Rutkoski, T, et al. Cancer Biology & Therapy 2011 12 (3): 208-214). With both RNase A and oligonucleotides functionalized on Au NPs, the nanozyme presented general cytotoxicity towards all 5 types of cells studied, with IC₅₀ around 10 nM throughout different types of cells. Such IC₅₀ was about two orders of magnitudes lower compared with that of reported toxic RNase, including onconase and other mutants of RNase A superfamily under similar conditions (Hoang, T, et al. Molecular Cancer Therapeutics 2018 17 (12): 2622-2632; Lee, I, et al. Anticancer Research 2007 27 (1A): 299-307; Shen, R, et al. Acta Biochim Biophys Sin (Shanghai) 2016 48 (10): 894-901), suggesting nanozymes' extremely high cytotoxicity. Additionally, it should be mentioned that with only RNase functionalized, the Au-LA-A19C NPs without oligonucleotides exhibited certain degree of cytotoxicity. For example, the IC₅₀ of Au-LA-A19C NPs with A549 cells were found to be 38.0 nM. But such cytotoxicity was much lower than that of nanozyme.

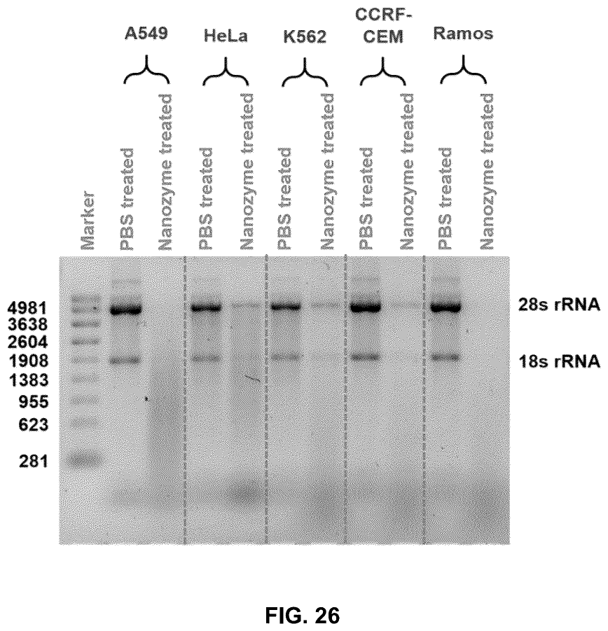

Next, nanozymes' effects on intracellular RNA among various types of cells were studied. Specifically, different types of cells were incubated with nanozyme at their IC₅₀ for 48 h and their intracellular RNA was analyzed by gel electrophoresis. As shown in FIG. 26, after incubation with nanozyme, similar intracellular 18s and 28s rRNA degradation was observed throughout all five types of cells, suggesting that nanozymes' general intracellular RNase activity on degrading 18s and 28s rRNA.

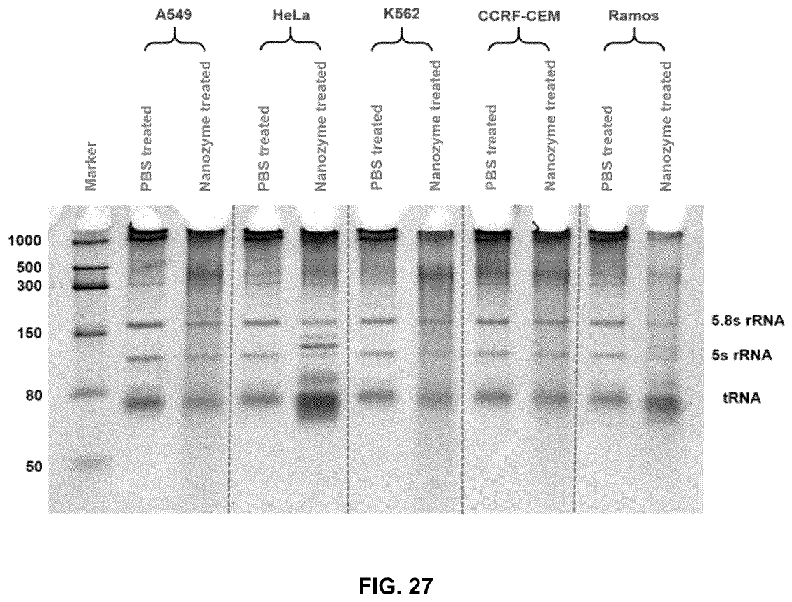

Meanwhile, nanozymes' effect on 5s, 5.8s rRNA and tRNA varied among different cell lines (FIG. 27), suggesting that different types of cells responded differently to the nanozyme treatment. But generally, the degradation of small sized 5s, 5.8s rRNA and tRNA were not as severe as that of larger sized 18s and 28s rRNA, which is worthy to be further investigated.

### Preparation and Characterization of Hollow Broad-Spectrum Nanozymes

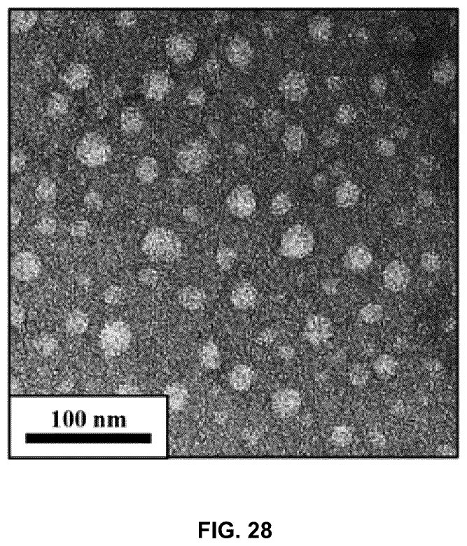

Following the method described above, hollow broad-spectrum nanozymes were synthesized using LA-A19C and capturer DNA modified with polymerizable oligos. After polymerization, the Au core was removed by KCN (0.05 M, 1 h) and purified by extensive dialysis. The structure of hollow broad-spectrum nanozymes were characterized by transmission electron microscope with uranium acetate negative staining (FIG. 28). With polymerized oligo shell, hollow broad-spectrum nanozymes showed an average diameter of about 35 nm. This is slightly larger than its size before KCN treatment, probably due to the structural swallowing without the inorganic core.

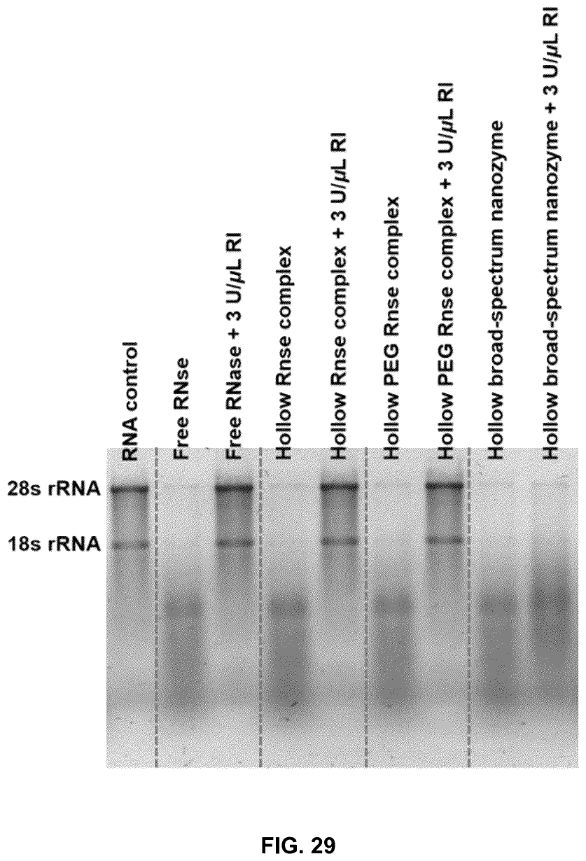

Electrophoresis analysis (FIG. 29) showed that hollow broad-spectrum nanozyme presented strong RNase activity both with and without RI, proving its excellent RI-resistance. On the contrary, the enzymatic activity of free RNase, hollow RNase complex and hollow PEG RNase complex are completed inhibited with the existence of RI.

TABLE 7
IC₅₀ of nanozyme, Au-poly A6 NPs, Au-LA-A19C NPs and free
RNase A, as well as hollow nanozyme, hollow-poly A6,
hollow LA-A19C NPs on various types of tumor cells.

| | A549 | | HeLa | | K562 | | CCRF-CEM | | Ramos | |
|---|---|---|---|---|---|---|---|---|---|---|
| Nanozyme | 10.7 | nM | 6.06 | nM | 13.3 | nM | 7.88 | nM | 7.68 | nM |
| Au-poly A₆ NPs | >40.0 | nM | >40.0 | nM | >40.0 | nM | >40.0 | nM | >40.0 | nM |
| Au-LA-A19C NPs | 38.0 | nM | 10.1 | nM | 24.6 | nM | 17.6 | nM | 16.8 | nM |
| Hollow nanozyme | 11.5 | nM | 5.02 | nM | 14.8 | nM | 10.3 | nM | 8.23 | nM |
| Hollow poly A₆ NPs | >40.0 | nM | >40.0 | nM | >40.0 | nM | >40.0 | nM | >40.0 | nM |
| Hollow LA-A19C NPs | >40.0 | nM | 13.9 | nM | 30.1 | nM | 20.9 | nM | 17.3 | nM |
| Free RNase A | >5.00 | μM | >5.00 | μM | >5.00 | μM | >5.00 | μM | >5.00 | μM |

Next, the cytotoxicity of hollow broad spectrum nanozyme were evaluated as IC₅₀ measurement towards A549, HeLa, K562, Ramos and CCRF-CEM Cells. As shown in Table 7, hollow broad-spectrum nanozyme presented strong cytotoxicity towards all types of cancer cell tested. Compared with broad-spectrum nanozyme with Au NPs core, hollow version of nanozyme without Au NPs cores exhibited similar IC₅₀ values. Such results suggested that, with densely crosslinked shell, the structurally integrative hollow broad-spectrum nanozyme possesses similar cytotoxicity as the ones with Au NPs as cores. As control groups, hollow poly A₆ NPs without enzyme showed no significant effect on cell viability, indicating that the cytotoxicity of densely crosslinked oligonucleotides is very low, which is in consistent with the data of Au-poly A₆ NPs. On the other hand, hollow-LA-A19C NPs without protective DNA exhibited certain degree of cytotoxicity. This is also in consistent with the data of Au-LA-A19C NPs, but such cytotoxicity was much lower than that of nanozyme.

### In Vitro Evaluation of Broad Spectrum Nanozymes and Hollow Broad-Spectrum Nanozymes' Antiviral Effect Toward Intracellular HCV RNA Replicon

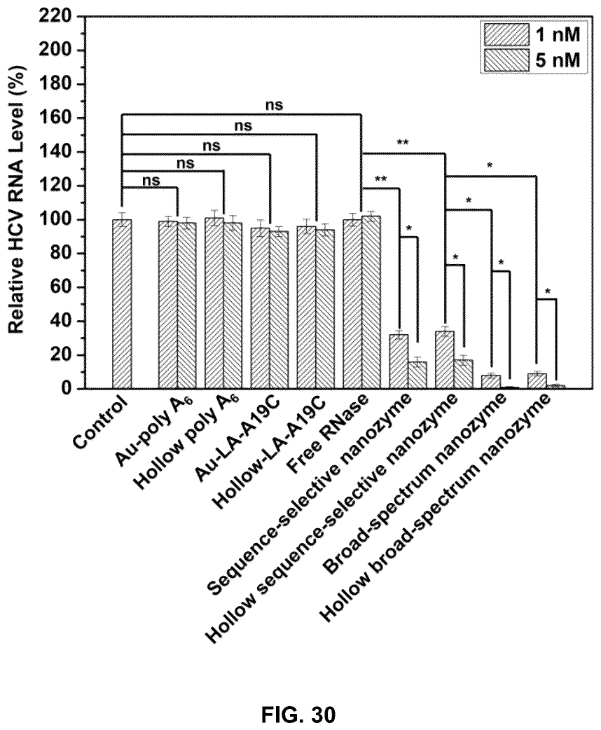

The antiviral efficacy of broad-spectrum nanozymes and hollow broad-spectrum nanozymes against hepatitis C virus was evaluated and compared with that of sequence-selective nanozyme we developed previously, based on mRNA level quantification using Quantitative real-time polymerase chain reaction (qRT-PCR) with the endogenous glyceraldehyde-3-phosphate dehydrogenase (GAPDH) gene as an internal standard. An HCV replicon cell culture system, a stable human hepatoma Huh7. 5-derived cell line harboring autonomously replicating genomic length genotype JFH1 HCV replicons is used. As shown in FIG. 30, no measurable reduction in the HCV RNA levels was observed in the treatments using Au-poly A₆, hollow poly A₆, Au-LA-A19C, hollow LA-A19C or free RNase at concentrations of 1 and 5 nM, suggesting that neither the assembly of oligonucleotides nor RNase possess any noticeable anti HCV effect under current testing conditions. On the contrary, when treated with broad-spectrum nanozyme or hollow broad-spectrum nanozyme under the same dosage, HCV replication in the cells dramatically decreased, and the inhibitory effect was dose dependent, proving that both broad-spectrum nanozyme or hollow broad-spectrum nanozyme possess excellent antiviral effect. Furthermore, compared with sequence-selective nanozyme developed previously, broad-spectrum nanozyme, whether with gold core or hollow, presented significantly higher antiviral efficiency. With 5 nM nanozyme dosage, cells treated by broad-spectrum nanozyme presented over 97% reduction in HCV mRNA level, suggesting that the replication of HCV replicon was almost completely shut down, whereas under identical testing conditions, only ˜80% mRNA reduction was observed.

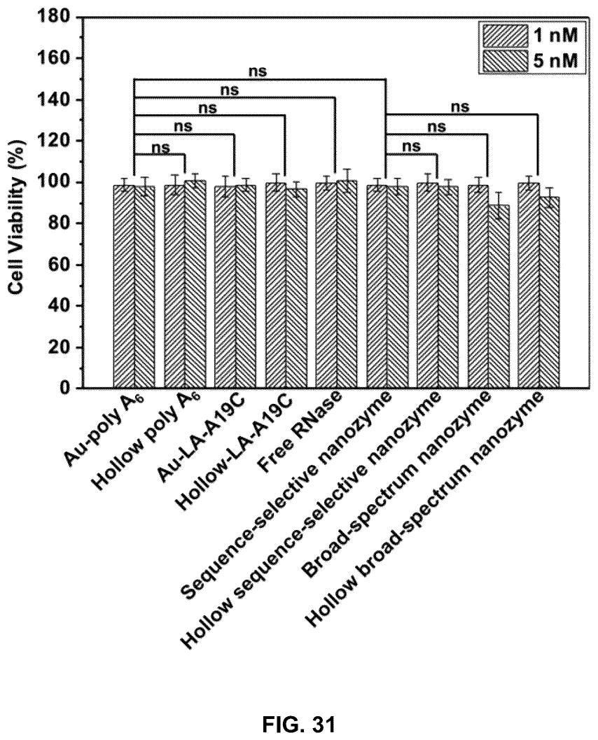

Moreover, as shown in FIG. 31, under testing nanozyme dosages, no obvious cytotoxicity was observed. Even under 5 nM broad-spectrum nanozyme treatment, over 90% cells viability was detected, thus suggesting the safety of broad-spectrum nanozyme as antiviral therapeutics.

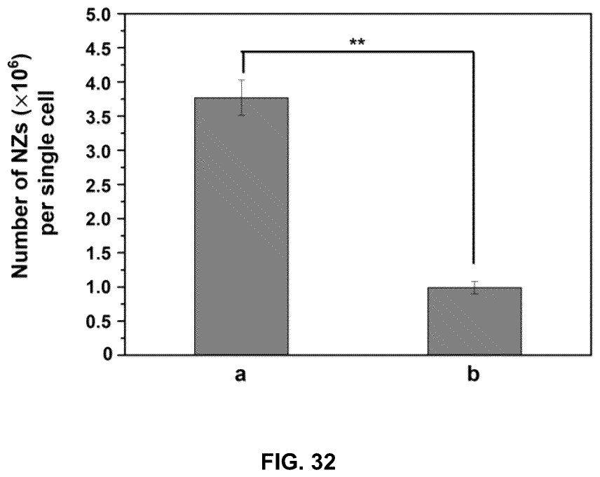

Such high potency of HCV mRNA down regulation was probably correlate with the efficient cellular uptake of broad-spectrum nanozyme. As shown in FIG. 32, compared with sequence-selective nanozyme, broad-spectrum nanozyme presented significantly higher cellular uptake, which was found to be almost 4 times as high as that of sequence-selective nanozyme. Indeed, RNase A was reported to exhibit highly efficient cellular entry through both clathrin-coated vesicles and macropinosomes, with efficiency comparable to cell penetrating peptide (Chao, T. Y, et al. Biochemistry 2011 50 (39): 8374-82; Haigis, M. C, et al. J Cell Sci 2003 116 (Pt 2): 313-24). With tens of RNase A molecules anchored on nanoscopic surface, such cell penetrating properties could probably be further amplified.

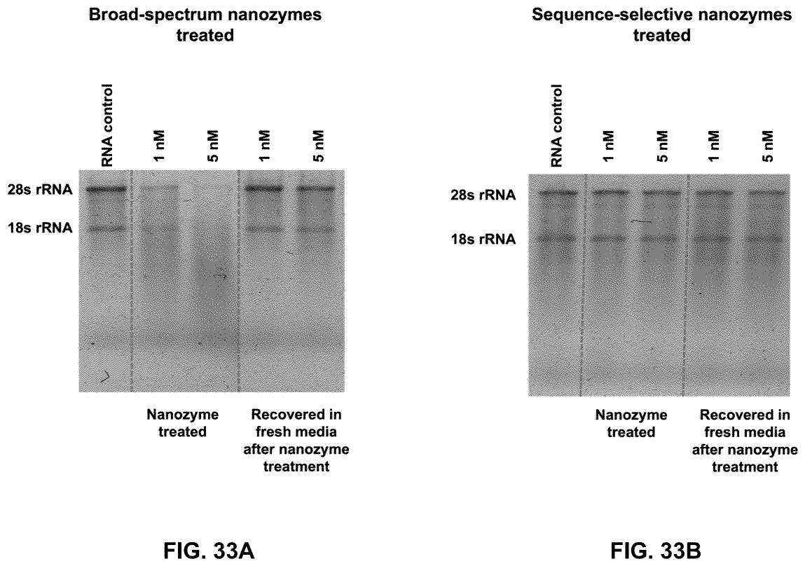

To better understand the mechanism of broad-spectrum nanozymes' anti-viral effect, the intracellular RNA of Huh7.5 cells harboring JFH1 HCV replicons treated with nanozymes was analyzed with gel electrophoresis. As shown in FIG. 33A, significant intracellular RNA degradation was observed for cells treated with broad-spectrum nanozymes, and more severe degradation was detected for cells treated with higher nanozyme dosages. However, once nanozymes-treated cells were re-cultured in fresh media without nanozyme, intracellular ribosomal RNA was found to be fully recovered. Therefore, it is expected that the antiviral effect of broad-spectrum nanozyme is likely achieved via a competitive mechanism, where endogenous RNAs can be regenerated from cellular genomic transcriptions, but exogenous RNAs cannot. This opens an opportunity of using broad-spectrum nanozymes as effective therapeutic agents to treat infections of RNA virus, such as HCV and COVID-19. As a comparison, cells treated with sequence-selective nanozyme exhibited no obvious intracellular RNA degradation (FIG. 33B).

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A nanozyme, comprising an engineered ribonuclease enzyme and a protective DNA oligonucleotide independently or collectively attached to a gold nanoparticle directly or indirectly by gold-sulfur bonds,
wherein the engineered ribonuclease enzyme comprises a mutated cysteine residue that is functionalized with a length-tunable multi-thiol tether,
wherein the protective DNA oligonucleotide is 1 to 22 nucleotides, and
wherein the nanozyme comprises a density of 20 to 100 DNA oligonucleotides and 30 to 60 engineered ribonuclease enzymes on the surface of the gold nanoparticle.

2. The nanozyme of claim 1, wherein the multi-thiol tether comprises is tagged with a lipoic acid moiety

3. The nanozyme of claim 1, wherein the multi-thiol tether comprises a polyethylene glycol spacer.

4. The nanozyme of claim 1, wherein the ratio of engineered ribonuclease enzymes to protective DNA oligonucleotides on the surface of the gold nanoparticle is from 1:3 to 1:0.5.

5. The nanozyme of claim 1, wherein the ribonuclease is ribonuclease is ribonuclease-A (RNase-A) or ribonuclease-1 (RNase-1).

6. The nanozyme of claim 5, wherein the engineered RNase-A enzyme comprises a cysteine substitution at amino acid residue A19, G88, or a combination thereof.

7. The nanozyme of claim 5, wherein the engineered RNase-1 enzyme comprises a cysteine substitution at amino acid residue P19, G89, or a combination thereof.

8. The nanozyme of claim 1, wherein the protective DNA oligonucleotide is thiol-modified and is directly attached to the gold nanoparticle by a gold-sulfur bond.

9. The nanozyme of claim 1, further comprising guiding DNA oligonucleotides, which can bind onto specific receptors on the surface of cells.

10. The nanozyme of claim 9, wherein the guiding DNA oligonucleotide is thiol-modified and is directly attached to the gold nanoparticle by a gold-sulfur bond.

11. A hollow nanozyme produced by a process comprising,
(a) affixing to a gold nanoparticle by gold-sulfur bonds, or affixing to an inorganic nanoparticle of other compositions with specific ligand-surface bonds
(i) 30 to 60 engineered ribonuclease enzymes comprising a mutated cysteine residue that is functionalized with multi-alkylthiol-terminated sequences of poly-thymine bases modified with propargyl ether, and
(ii) 30 to 100 alkylthiol-terminated and propargyl-ether-modified protective DNA oligonucleotide 1 to 22 nucleotides in length;
(b) polymerizing the propargyl ether groups; and
(c) removing the inorganic nanoparticle with chemicals that can dissolve the particle, thereby producing a hollow nanoenzyme.

12. The hollow nanozyme of claim 12, wherein step (c) comprises removing the gold nanoparticle with potassium cyanide.

13. The hollow nanozyme of claim 12, wherein the ratio of engineered ribonuclease enzymes to protective DNA oligonucleotides on the surface of the gold nanoparticle is from 1:3 to 1:0.5.

14. The hollow nanozyme of claim 12, wherein the ribonuclease is ribonuclease-A (RNase-A) or ribonuclease-1 (RNase-1).

15. The hollow nanozyme of claim 15, wherein the engineered RNase-A enzyme comprises a cysteine substitution at amino acid residue A19, G88, or a combination thereof.

16. The hollow nanozyme of claim 15, wherein the engineered RNase-1 enzyme comprises a cysteine substitution at amino acid residue P19, G89, or a combination thereof.

17. The hollow nanozyme of claim 12, further comprising a guiding DNA oligonucleotide.

18. The hollow nanozyme of claim 17, wherein the guiding DNA oligonucleotide comprises a first RNA recognition moiety comprising a nucleic acid sequence complementary to a first region of an RNA target, wherein the RNA target is cleaved by the recombinant RNase enzyme when the first RNA recognition moiety binds to the RNA target.

19. The hollow nanozyme of claim 12, further comprising drug molecules encapsulated within the nanozyme.
A method for silencing RNA in a cell, comprising contacting the cell with the nanozyme of claims 1 to 19.


SEQUENCE LISTING (8 sequences)

SEQ ID NO: 1 - protein, 124 aa, unidentified
KETAAAKFERQHMDSSTSAASSSNYCNQMMKSRNLTKDRCKPVNTFVHESLADVQAVCSQKNVACKNGQTNCYQSYSTMSITDCRETGSSKYPNCAYKTTQANKHIIVACEGNPYVPVHFDASV

SEQ ID NO: 2 - protein, 124 aa, unidentified
KETAAAKFERQHMDSSTSCASSSNYCNQMMKSRNLTKDRCKPVNTFVHESLADVQAVCSQKNVACKNGQTNCYQSYSTMSITDCRETGSSKYPNCAYKTTQANKHIIVACEGNPYVPVHFDASV

SEQ ID NO: 3 - protein, 124 aa, unidentified
KETAAAKFERQHMDSSTSAASSSNYCNQMMKSRNLTKDRCKPVNTFVHESLADVQAVCSQKNVACKNGQTNCYQSYSTMSITDCRETCSSKYPNCAYKTTQANKHIIVACEGNPYVPVHFDASV

SEQ ID NO: 4 - protein, 128 aa, unidentified
KESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 5 - protein, 128 aa, unidentified
KESRAKKFQRQHMDSDSSCSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 6 - protein, 128 aa, unidentified
KESRAKKFQRQHMDSDSSPSSSSTYCNQMMRRRNMTQGRCKPVNTFVHEPLVDVQNVCFQEKVTCKNGQGNCYKSNSSMHITDCRLTNCSRYPNCAYRTSPKERHIIVACEGSPYVPVHFDASVEDST

SEQ ID NO: 7 - DNA, 18 bp, unidentified
  source 1..18: mol_type = other DNA
cttgaatgtagagatgcg

SEQ ID NO: 8 - DNA, 39 bp, unidentified
  source 1..39: mol_type = other DNA
ttttggtttttctttgaggtttaggatttgtgctcatgg